(19) United States
(12) Patent Application Publication
(10) Pub. No.: US 2023/0104671 A1
(43) Pub. Date: Apr. 6, 2023

(54) METHODS AND COMPOSITIONS FOR TREATING CARDIOMYOPATHY AND HEART FAILURE

(71) Applicant: Ann and Robert H. Lurie Children's Hospital of Chicago, Chicago, IL (US)

(72) Inventor: Youyang Zhao, Deerfield, IL (US)

(21) Appl. No.: 17/937,758

(22) Filed: Oct. 3, 2022

Related U.S. Application Data
(60) Provisional application No. 63/262,008, filed on Oct. 1, 2021.

(51) Int. Cl. A61K 31/4709; A61K 31/4706; A61K 31/352; A61K 31/277; A61K 31/198; A61P 9/04

(52) U.S. Cl. CPC A61K 31/4709; A61K 31/4706; A61K 31/352; A61K 31/277; A61K 31/198; A61P 9/04

(57) ABSTRACT

Disclosed are methods and compositions for treating cardiovascular diseases including cardiomyopathy and heart failure. Particularly disclosed are methods and compositions that utilize or comprise inhibitors of hypoxia-inducible factor (HIF)-2α or agonists/inducers of HIF prolyl hydroxylase domain-2 (PHD2) signaling for treating cardiomyopathy and heart failure.

### CROSS-REFERENCE TO RELATED APPLICATIONS

The present application claims priority to U.S. Provisional Patent Application No. 63/262,008, filed Oct. 1, 2021, the entire contents of which are hereby incorporated by reference.

### STATEMENT REGARDING FEDERALLY SPONSORED RESEARCH

This invention was made with government support under HL148810, HL140409, and HL133851 awarded by the National Institutes of Health. The government has certain rights in the invention.

### REFERENCE TO AN ELECTRONIC SEQUENCE LISTING

The contents of the electronic sequence listing (702581.02232.xml; Size: 11,485 bytes; and Date of Creation: Oct. 3, 2022) is herein incorporated by reference in its entirety.

### FIELD OF THE INVENTION

The field of the invention relates to methods and compositions for treating cardiovascular diseases including cardiomyopathy and heart failure. In particular, the field of the invention relates to methods and compositions that utilize or comprise inhibitors of hypoxia-inducible factor (HIF)-2α or prolyl hydroxylase domain-2 (PHD2) agonists or inducers for treating cardiomyopathy and heart failure.

### BACKGROUND OF THE INVENTION

During development, the heart grows by cardiomyocyte proliferation and hypertrophy while after birth, the cardiomyocytes lose proliferative potential and heart growth is mainly via cardiomyocyte hypertrophy. Cardiac hypertrophy can happen in physiological and pathophysiological conditions. Pathological hypertrophy induced by hypertension, myocardial infarction, and/or cardiomyopathy results in ventricular remodeling which is associated with systolic and diastolic dysfunction and interstitial fibrosis, and finally leads to deleterious outcomes such as heart failure^{1,2}.

There are multiple cell types in the heart including cardiomyocytes, endothelial cells (ECs), fibroblasts and smooth muscle cells. ECs lining of the inner layer of the blood vessels account for the greatest number of cells in the heart. One of the major functions of cardiac vascular ECs is to supply oxygen and nutrients to support cardiomyocytes^{3,4}. Previous studies have demonstrated that angiogenesis stimulated by VEGF-B or PIGF induces a marked increase of cardiac mass in rodents, whereas inhibition of angiogenesis results in decreased capillary density, contractile dysfunction, and impaired cardiac growth^{5,6}. Cardiac vasculature rarefaction is associated with pathological cardiac hypertrophy and heart failure⁷. Recent studies have also demonstrated the important role of EC-derived paracrine factors such as Endothelin-1, Apelin, Neuregulin and Agrin in regulating cardiac hypertrophy, regeneration, and repair^{8,9}. However, how ECs crosstalk with cardiomyocytes in the pathogenesis of cardiac hypertrophy and dysfunction is not fully understood.

### BRIEF SUMMARY OF THE INVENTION

Disclosed herein are compositions and methods useful for the treatment of cardiovascular disease, including cardiomyopathy, heart failure, or hypertrophy, in a subject in need thereof. In some embodiments the methods comprise administering to the subject in need an effective amount of a HIF-2α inhibitor. In particular embodiments, the HIF-2α inhibitor includes ethacridine, quinidine or an analog of quinidine such as hydroxyethylapoquinine (HEAQ), methantheline, propantheline, or their analogs or any combination thereof. In particular embodiments, the HIF-2α inhibitor includes PT2977, PT2385, PT2399, NKT2152 or C76 or their analogs or any combination thereof.

Pharmaceutical compositions for use in the treatment of cardiovascular disease, including cardiomyopathy, heart failure, and hypertrophy, may include a HIF-2α inhibitor. In particular embodiments, the HIF-2α inhibitor includes ethacridine, quinidine or an analog of quinidine such as hydroxyethylapoquinine (HEAQ), methantheline, propantheline, or their analogs or any combination thereof. In particular embodiments, the HIF-2α inhibitor includes PT2977, PT2385, PT2399, NKT2152 or C76 or their analogs or any combination thereof. In some embodiments, the HIF-2α inhibitor is PHD2. In some embodiments, the method further comprising administering an agent to activate PHD2 or induce PHD2 expression.

Another aspect of the invention provides for a method for treating cardiovascular disease, including cardiomyopathy, heart failure, or hypertrophy, in a subject by administering an effective amount of an agent to activate PHD2 or to induce PHD2 expression.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Decreased PHD2 expression in heart vascular ECs of patients with cardiomyopathy. (A) Single-cell RNA sequencing analysis from human fetal hearts showed that cardiac ECs express higher levels of EGLN1, EPAS1 compared to other cell types. Mac=macrophage, SMC=smooth muscle cell, Fib=fibroblast, CM=cardiomyocyte. (B) QRT-PCR analysis showing that EGLN1 mRNA levels in LV hearts were significantly downregulated in dilated cardiomyopathy (DCM) patients compared to healthy donors. N=3 each group. **p<0.01 (Student's t test). (C and D) Immunohistochemistry of PHD2 (C) and quantification (D) demonstrating a significant decrease of PHD2 expression in LV sections, especially in vascular endothelial cells (ECs) of patients with cardiomyopathy (CM). Immunostaining intensity was graded from 1 to 10, with 10 being the highest. White dots indicate hypertrophic cardiomyopathy, red dot indicates dilated cardiomyopathy, green dot indicates hypertensive cardiomyopathy. Scale bar, 50 mm. *p<0.05 (linear regression analysis with consideration of age as a covariate).

FIG. 2. Single-cell RNA sequencing analysis of human fetal hearts. (A) A UMAP plot showing the major cardiac cell types including ECs, cardiomyocytes (CM), fibroblasts (Fib), smooth muscle cells (SMC) and macrophages (Mac). (B) A Violin plot showing the expression levels of PHD2/HIF signaling genes in cardiac cells.

FIG. 3. Constitutive loss of endothelial Egln1 leads to left ventricular hypertrophy and fibrosis. (A) Increased weight ratio of LV/body weight of Egln1^{Tie2Cre} (CKO) mice compared to WT mice at various ages. M=month. (B and C) Representative M model of echocardiography measurement showing that Egln1^{Tie2Cre} mice exhibit increase of LV anterior, posterior wall thicknesses, and reduction of LV internal dimension (LVID) compared to WT mice at the age of 3.5 months. (D and E) Quantification of LV anterior and posterior wall thicknesses via echocardiography measurement WT and Egln1^{Tie2Cre} mice. (F) H&E staining showing cardiac hypertrophy in Egln1^{Tie2Cre} mice at the age of 3.5 months. Scale bar, 1 mm. (G) WGA staining of cardiac sections and quantification demonstrating enlargement of cardiomyocytes in Egln1^{Tie2Cre} mice. N=5/group. Scale bar, 20 mm. (H) Trichrome staining showing prominent cardiac fibrosis in Egln1^{Tie2Cre} mice. Scale bar, 100 mm. *p<0.05, **p<0.01, ***p<0.001 (Student's t test).

FIG. 4. Upregulation of fetal gene including Bnp and fibrotic gene Col1a in the LV of Egln1^{Tie2Cre} mice. *p<0.05, **p<0.01 (Student's t test).

FIG. 5. Egln1 deficiency in bone marrow cells does not contribute to cardiac hypertrophy. (A) A diagram showing the strategy of bone marrow cell transplantation. Lethally gamma-irradiated WT were transplanted with bone marrow (BM) cells freshly isolated from WT or Egln1^{Tie2Cre} (CKO) mice. Similarly, irradiated CKO mice were reconstituted with WT or CKO bone marrow cells. (B) Cardiac dissection showed that Egln1-deficient bone marrow cells did not contribute to cardiac hypertrophy seen in Egln1^{Tie2Cre} (CKO) mice.

FIG. 6. Inducible deletion of endothelial Egln1 in adult mice leads to left ventricular hypertrophy and fibrosis. (A) A diagram showing the strategy of generating Egln1^{EndoCreERT2} mice (iCKO). (B) Egln1^{EndoCreERT2} mice exhibited increase of LV/body weight ratio compared to age-matched WT mice at ˜7 months post-tamoxifen treatment. (C) Representative M model of echocardiography showing LV hypertrophy in Egln1^{EndoCreERT2} mice. (D and E) Quantification of LVAWd and LVPWd showing increased LV wall thicknesses of Egln1^{EndoCreERT2} mice. (F) H&E staining demonstrating that Egln1^{EndoCreERT2} mice developed LV hypertrophy. Scale bar, 1 mm. (G) Trichrome staining showing the deposition of collagen in the LV of Egln1^{EndoCreERT2} mice. Scale bar, 100 mm. **p<0.01, ***p<0.001 (Student's t test).

FIG. 7. Deletion of endothelial Egln1 increased angiogenesis in the left ventricles. (A and B) Immunostaining of CD31 and WGA and quantification showing increase of capillary EC versus cardiomyocyte number in Egln1^{Tie2Cre} mice. Left heart sections were stained with anti-CD31 (red, marker for ECs) and WGA (green). N=4/group. Scale bar, 20 mm. (C and D) Anti-Ki67 staining, and quantification demonstrated that cardiac EC was hyperproliferative in the LV of Egln1^{Tie2Cre} mice. Left heart sections were immunostained with anti-Ki67 (red, cell proliferation marker) and anti-CD31 (green). Nuclei were counterstained with DAPI. N=4/group. Scale bar, 50 mm. **p<0.01, ***p<0.001 (Student's t test).

FIG. 8. Distinct role of endothelial HIF-1a and HIF-2a in Egln1 deficiency-induced left heart hypertrophy and fibrosis. (A) A diagram demonstrating generation of Egln1/Hif2a^{Tie2Cre} (EH2), Egln1/Hif1a^{Tie2Cre} (EH1) and Egln1/Hif1a/Hif2a^{Tie2Cre} (EH1/2) mice. (B) Cardiac dissection showing that endothelial HIF-2a deletion protected from endothelial Egln1 deficiency-induced LV hypertrophy, whereas HIF-1α deletion augmented LV hypertrophy. (C and D) Echocardiography demonstrated that HIF-2a deletion in ECs protected from LV wall thickening induced by Egln1 deficiency. (E) H&E staining showed normalization of LV hypertrophy in Egln1/Hif2a^{Tie2Cre} mice. Scale bar, 1 mm. (F) WGA staining and quantification showed a complete normalization of cardiomyocyte hypertrophy in Egln1/Hif2a^{Tie2Cre} mice. The same surface area data of WT and KO in FIG. 2H was used. N=5/group. Scale bar, 20 mm. (G) Trichrome staining demonstrated absence of collagen deposition in the LV of Egln1/Hif2a^{Tie2Cre} mice. Scale bar, 100 mm. *p<0.05, **p<0.01, ***p<0.001 (One-way ANOVA with Tukey post hoc analysis for multiple group comparisons).

FIG. 9. RNA-sequencing analysis identifies multiple dysfunctional pathways in regulation of cardiac hypertrophy and fibrosis induced by endothelial Egln1 deficiency and normalization by HIF-2a disruption. (A) A representative heatmap of RNA-sequencing analysis of WT, Egln1^{Tie2Cre} (CKO) and Egln1/Hif2a^{Tie2Cre} (EH2) mice. (B) KEGG pathway enrichment analysis of upregulated genes in Egln1^{Tie2Cre} vs WT mice showing dysregulation of multiple signaling pathways related to cardiac hypertrophy. (C) KEGG pathway enrichment analysis indicates that HIF-2α-activated downstream genes are related to hypertrophic cardiomyopathy. (D) A heatmap showing the expression of genes related to endothelium and endothelial cell-derived factors-mediated cell growth, genes related to cardiac fibrosis, and HIF target genes.

FIG. 10. Pharmacological inhibition of HIF-2a attenuated cardiac hypertrophy and fibrosis. (A) Inhibition of HIF-2a reduced weight ratio of LV/body weight (BW) in Egln1^{Tie2Cre} mice. (B and C) WGA staining and quantification demonstrated that HIF-2a inhibition reduced cardiomyocyte hypertrophy. N=5/group. Scale bar, 20 mm. (D and E) Trichrome staining revealed attenuation of cardiac fibrosis by HIF-2a inhibition. Scale bar, 100 mm. *p<0.05, **p<0.01, p<0.001 (Student's t test).

### DETAILED DESCRIPTION OF THE INVENTION

Cardiovascular diseases, including cardiomyopathy and hypertrophy are common adaptive responses to injury and stress. Hypoxia signaling is important to the (patho)physiological process of cardiac remodeling. However, the role of endothelial Prolyl-4 hydroxylase 2 (PHD2)/hypoxia inducible factors (HIFs) signaling in the pathogenesis of cardiac hypertrophy and heart failure is unknown. Presented herein is an unexpected role of endothelial PHD2 deficiency in inducing cardiac hypertrophy and fibrosis in a HIF-2a dependent manner.

Compositions and methods for treating cardiovascular disease, including cardiomyopathy and hypertrophy in a subject in need thereof with compounds disclosed herein are provided. As used herein, a “subject” may be interchangeable with “patient” or “individual” and means an animal, which may be a human or non-human animal, in need of treatment. In particular embodiments, the subject is a human subject.

A “subject in need of treatment” may include a subject having a disease, disorder, or condition that may be characterized as a cardiovascular disease, including but not limited to cardiomyopathy, left ventricular hypertrophy or heart failure.

Cardiomyopathy represents a collection of diverse conditions of the heart muscle. Cardiomyopathy is a disease of the heart muscle that makes it harder for the heart to pump blood to the rest of the body and can lead to heart failure. Cardiomyopathy can be acquired, e.g., developed because of another disease, condition, or factor, or inherited. The cause is not always known. The main types of cardiomyopathy include dilated, hypertrophic, arrhythmogenic and restrictive cardiomyopathy.

Left ventricular hypertrophy (LV hypertrophy or LVH) is a thickening of the walls of the lower left heart chamber. The left ventricle is the heart's main pumping chamber and this chamber pumps oxygenated blood into the aorta, the large blood vessel that delivers blood to the body's tissues. If the left ventricle has to work too hard, its muscle hypertrophies (enlarges) and becomes thick. This is called left ventricular hypertrophy (LVH). The thickened heart wall can become stiff and blood pressure in the heart increases, making it harder for the heart to effectively pump blood. Because of the increased thickness, blood supply to the muscle itself may become inadequate. This can lead to cardiac ischemia (not enough blood and oxygen at the tissue level), myocardial infarction (heart attack), or heart failure. Uncontrolled high blood pressure is the most common cause of LV hypertrophy. Complications include irregular heart rhythms, called arrhythmias, and heart failure.

Heart failure occurs when the heart muscle doesn't pump blood as well as it should. Heart failure develops when the heart, via an abnormality of cardiac function (detectable or not), fails to pump blood at a rate commensurate with the requirements of the metabolizing tissues or is able to do so only with an elevated diastolic filling pressure. Heart failure can occur if the heart cannot pump (systolic) or fill (diastolic) adequately. Congestive heart failure is a type of heart failure.

The Examples show that cardiomyopathy patients have decreased levels of PHD2, and that loss of endothelial PHD2 induces LV hypertrophy, including increased LV wall thickness, reduced LV chamber size, and increased expression of hypertrophy markers including the genes Ano, Bnp and Myh7. Further, a causal role for endothelial HIF-2a in mediating LV hypertrophy and fibrosis is shown. Thus compositions that inhibit HIF-2a or increase PHD2 may be therapeutic in cardiomyopathy.

In some embodiments, a subject may be in need of treatment, for example, treatment may include administering a therapeutic amount of one or more agents that inhibits, alleviates, or reduces the signs or symptoms of cardiomyopathy.

Pharmaceutical or therapeutic compositions disclosed herein may be formulated for administration by any suitable route of delivery, such as oral, parenteral, rectal, nasal, topical, or ocular routes, or by inhalation.

In some embodiments the compositions and methods for treating cardiovascular disease, including cardiomyopathy and LV hypertrophy comprises administering effective amount of an inhibitor of hypoxia-inducible factor (HIF)-2a (HIF-2a). HIFs are transcription factors that respond to decreases in available oxygen in the cellular environment, or hypoxia. Members of the HIF family include HIF-1α, HIF-1β, HIF-2α, and HIF-3α. The α-subunit of HIF is delicately controlled by HIF-prolyl hydroxylases (PHD) 1-3.

HIF-2α (or HIF-2alpha or HIF-2a) is, encoded by HIF2A, the endothelial PAS domain-containing protein 1 in mammals. HIF-2α is active under hypoxic conditions and is important for maintaining the catecholamine balance required for protection of the heart. Hypoxia-inducible factor prolyl hydroxylase 2 (HIF-PH2), or prolyl hydroxylase domain-containing protein 2 (PHD2), is an enzyme encoded by the EGLN1 gene. It is also known as Egl nine homolog 1. PHD2 is a α-ketoglutarate/2-oxoglutarate-dependent hydroxylase, a superfamily non-heme iron-containing proteins. In humans, PHD2 is one of the three isoforms of hypoxia-inducible factor-proline dioxygenase, which is also known as HIF prolyl-hydroxylase.

Under hypoxia condition, PHD activities are inhibited leading to stabilization of HIF-α proteins which activate downstream target gene expression. In normoxia, HIF alpha subunits are marked for the ubiquitin-proteasome degradation pathway through hydroxylation of proline-564 and proline-402 by PHD2. Both HIF-1α and HIF-2α share similar DNA binding site or hypoxia response element (HRE). Thus, some genes are co-regulated by HIF-1α and HIF-2α. However, HIF-1α and HIF-2α also control some sets of unique downstream targets, for example, LDHA, is a HIF-1α target while OCT4 is a HIF-2α target.

The pharmaceutical compositions of the present disclosure may include one or more compounds that inhibit HIF-2α. By way of example, but not by way of limitation, such compounds include quinidine and analogs thereof, and ethacridine and analogs thereof, propantheline/methantheline and analogs thereof. Additional HIF-2α inhibitors include, without limitation, PT2977 (also known as belzutifan, Welireg, MK-6482), PT2385, PT2399, NKT2152, C76, and analogs thereof. PHD2 also inhibits HIF2a through hydroxylation of proline-564 and proline-402 and thereby marking HIF for the ubiquitin-proteasome degradation pathway. In some embodiments, the pharmaceutical composition of the present disclosure includes one or more HIF-2α inhibitors wherein at least one of the HIF-2α inhibitors is PHD2.

As used herein the term “analogue” or “functional analogue” refer to compounds having similar physical, chemical, biochemical, or pharmacological properties. Functional analogues are not necessarily structural analogues with a similar chemical structure. An example of pharmacological functional analogues are morphine, heroine, and fentanyl, which have the same mechanism of action, but fentanyl is structurally quite different from the other two.

Quinidine (CAS 56-54-2) is a medication that acts as a class I antiarrhythmic agent in the heart. It prolongs cellular action potential and decreases automaticity. Quinidine also blocks muscarinic and alpha-adrenergic neurotransmission. Analogs of quinidine include, but are not limited to hydroxyethylapoquinine (1-[(5Z)-5-ethylidene-1-azabicyclo[2.2.2]octan-2-yl]-1-(6-hydroxyquinolin-4-yl)propane-1,3-diol; dihydrochloride; CAS 5414-52-8; HEAQ), hydroxyethylquinine (HEQ) [Antimicrob Agents Chemother. 2014 February; 58(2): 820-827], and hydroxyethylquinidine (HEQD)[Id.].

Ethacridine (7-ethoxyacridine-3,9-diamine, CAS 442-16-0) is an aromatic organic compound based on acridine. Its primary use is as an antiseptic in solutions of 0.1%. It is effective against mostly Gram-positive bacteria, such as Streptococci and Staphylococci, but ineffective against Gram-negative bacteria such as Pseudomonas aeruginosa. Ethacridine is also used as an agent for second trimester abortion.

Propantheline (methyl-di(propan-2-yl)-[2-(9H-xanthene-9-carbonyloxy)ethyl]azanium, brand name Probanthine among others; CAS 298-50-0) is one of a group of antimuscarinic agents (including methantheline bromide) for the treatment of excessive sweating, cramps or spasms of the stomach, intestines (gut) or bladder, and involuntary urination. It can also be used to control the symptoms of irritable bowel syndrome and similar conditions.

Methantheline (diethyl-methyl-[2-(9H-xanthene-9-carbonyloxy)ethyl]azanium; CAS 5818-17-7) is a synthetic quaternary ammonium antimuscarinic used to relieve cramps or spasms of the stomach, intestines, and bladder.

PT2977 (3-[[(1S,2S,3R)-2,3-difluoro-1-hydroxy-7-methylsulfonyl-2,3-dihydro-1H-inden-4-yl]oxy]-5-fluorobenzonitrile; CAS 1672668-24-4), also known as Belzutifan or MK-6482, the brand name Welireg, is an orally active, small molecule inhibitor of hypoxia inducible factor HIF-2a with potential antineoplastic activity. Upon oral administration, belzutifan binds to and blocks the function of HIF-2α, thereby preventing HIF-2α/HIF-β heterodimerization and its subsequent binding to DNA. This results in decreased transcription and expression of HIF-2α downstream target genes, many of which regulate hypoxic signaling.

PT2385 (3-[[(1S)-2,2-difluoro-1-hydroxy-7-methyl sulfonyl-1,3-dihydroinden-4-yl]oxy]-5-fluorobenzonitrile; CAS 1672665-49-4) is a HIF-2α inhibitor which causes an allosteric disruption of a protein-protein interaction between HIF-2α and Aryl Hydrocarbon Receptor Nuclear Translocator (ARNT).

PT2399 (3-[[(1S)-7-(difluoromethylsulfonyl)-2,2-difluoro-1-hydroxy-1,3-dihydroinden-4-yl]oxy]-5-fluorobenzonitrile; CAS 1672662-14-4) is a selective HIF-2α antagonist, which directly binds to HIF-2α PAS B domain.

NKT2152 (CAS 2511247-29-1) is a selective HIF-2α inhibitor that blocks HIF herterodimerization and inhibits HIF-2α function. [WO 2022/187528; NCT05119335]

Compound 76 or C76 (methyl 3-[(2E)-2-[cyano(methylsulfonyl)methylidene]hydrazinyl]thiophene-2-carboxylate; CAS 882268-69-1) is a cell-permeable thienylhydrazone compound that suppresses the translation of cellular HIF-2α message by enhancing the binding of IRP1 (Iron-Regulatory Protein 1) to the IRE (Iron-Responsive Element) region at the 5′ UTR of the HIF-2α mRNA.

In some embodiments, pharmaceutical composition of the present disclosure includes one or more HIF-2α inhibitors, and at least one additional active agent or procedure. KRH102053 (4-methoxy-6-((4-methoxybenzyl)amino)-2,2-dimethylchroman-3-yl methioninate; CAS 1353254-53-1) or analogs thereof or KRH102140 (N-(2-fluorobenzyl)-2-methyl-2-phenethyl-2H-chromen-6-amine; CAS 864769-01-7) are exemplary molecules that activate PHD2 and inhibit HIF activity.

Exemplary additional active agents and procedures include but are not limited to angiotensin-converting enzyme inhibitors, angiotensin II receptor blockers, beta blockers, calcium channel blockers, water pills, also called diuretics, catheter procedure, and surgery.

Use of pharmaceutically acceptable inert carriers to formulate the compounds herein disclosed for the practice of the disclosure into dosages suitable for administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, dragees, liquids, gels, syrups, slurries, suspensions, and the like, for oral ingestion.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In some embodiments, the subject is responsive to therapy with one or more of the compounds disclosed herein in combination with one or more additional therapeutic agents.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be improving symptoms of cardiomyopathy.

In some embodiments, improvement in a patient's condition, as compared to an untreated control subject, is noted within about a day, a week, two weeks, within about one month or two months after the first treatment is administered. By way of example, improvements in a patient's condition may include, but is not limited to an improvement in clinical symptoms, test results, histopathological findings, quality of life, LV wall thickness, LV chamber size, LV cell surface measurements, and perivascular or intercardiomyocyte fibrosis.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Some embodiments of the present disclosure include methods for treating cardiovascular disease, including cardiomyopathy, LV hypertrophy and heart failure. In some embodiments, the cardiovascular disease is characterized by decreased expression of PHD2 in endothelial cells.

In some embodiments the methods for treating cardiovascular disease include administration to a subject, an effective amount of a HIF-2α inhibitor. Without limitation, the method may include administering ethacridine, quinidine or an analog of quinidine such as hydroxyethylapoquinine (HEAQ), methantheline, and propantheline, and their analogs. Without limitation, the method may include administering PT2977, PT2385, PT2399, NKT2152, C76, combinations thereof or analogs thereof. In particular embodiments, the method may include increasing PHD2 activity or expression.

Some embodiments of the present disclosure include methods for reducing left ventricular hypertrophy wherein the method comprises administering an effective amount of a HIF-2α inhibitor. Without limitation, the method may include administering ethacridine, quinidine or an analog of quinidine such as hydroxyethylapoquinine (HEAD), methantheline, and propantheline, and their analogs. Without limitation, the method may include administering PT2977 (belzutifan Welireg, MK-6482), PT2977, PT2385, PT2399, NKT2152, C76, combinations thereof or analogs thereof. In particular embodiments, the method may include increasing PHD2 activity or expression.

Some embodiments of the present disclosure include methods comprising administering an effective amount of an agent to activate PHD2 or to induce PHD2 expression. Activating or inducing PHD2 causes the transcription or translation of the EGLN1 gene, or alternatively an active form of the PHD2 protein. Based on the Example herein, PHD2 is active under normoxic conditions and PHD2 expression is decreased in cardiomyopathy patients. An agent to activate PHD2 or induce PHD2 expression causes a change in EGLN1 gene expression or PHD2 protein such that there is more EGLN1 or PHD2 protein activity is increased.

Unless defined otherwise, all technical and scientific terms used herein have the same meaning as is commonly understood by one of ordinary skill in the art to which this invention belongs.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

As used herein, the term “decrease” or the related terms “decreased,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

As used herein, the term “increase” or related terms means a change or combination of changes that result in a second measurement exceeding the first measurement.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

Methods and Compositions for Treatment of Cardiomyopathy and Heart Failure

Hypoxia-inducible factors (HIF) are key transcriptional factors mediating cellular response to oxygen levels. The a-subunit of HIF is delicately controlled by HIF-prolyl hydroxylases (PHD 1-3)¹⁰⁻¹². Under normoxia condition, PHDs hydroxylate HIF-α (mainly HIF-1α and HIF-2α), then E3 ligase VHL promotes degradation of hydroxylated HIF-α through proteasome-degradation pathway. Under hypoxia condition, PHD activities are inhibited leading to stabilization of HIF-α proteins which activate downstream target gene expression. Both HIF-1α and HIF-2α share similar DNA binding site or hypoxia response element (HRE)^{11,13}. Thus, some genes are co-regulated by HIF-1α and HIF-2α, such as CXCL12. However, HIF-1α and HIF-2α also control some sets of unique downstream targets, for example, LDHA, is a HIF-1α target while OCT4 is a HIF-2α target¹⁴. HIF-1α is ubiquitously expressed whereas HIF-2α is more restricted in certain cell types such as endothelial cells (ECs), and alveolar type 2 epithelial cells¹⁵, suggesting that HIF-α have distinct function in different cell types under different (patho)physiological conditions.

Egln1 null mutant mice exhibit polycythemia and congestive heart failure¹⁸. However, cardiomyocyte specific-disruption of Egln1 causes only mild abnormality with the presence of occasional myocytes with increased hypereosinophilia and blurring of the cross-striations¹⁹, indicating loss of PHD2 cells other than cardiomyocytes causes congestive heart failure. Fan et al. show that EC deletion of both PHD2 and 3 induces spontaneous cardiomegaly due to enhanced cardiomyocyte proliferation and also prevents heart failure induced by myocardial infarction. However, it is unknown whether these phenotypes are mediated by endothelial loss of either PHD2 or PHD3 alone. Studies have shown loss of both PHD2 and 3 but not PHD2 alone in cardiomyocyte induces ischemic cardiomyopathy²⁰. To our surprise, we observed spontaneous left ventricular (LV) hypertrophy and cardiac fibrosis in tamoxifen-inducible EC-specific Egln1 knockout adult mice. Using the mice with endothelial deletion of Egln1 alone, Egln1 and Hif1a, or Egln1 and Hif2a, we found that loss of endothelial PHD2-induced cardiac hypertrophy and fibrosis was mediated by HIF-2α activation and pharmacological inhibition of HIF-2α inhibited LV hypertrophy and cardiac fibrosis. Analysis of single cell RNA sequencing datasets revealed EGLN1 mainly expressed in vascular ECs of the human heart under normal condition. In patients with hypertrophic cardiomyopathy, EGLN1 expression in LV heart tissue (mRNA) and cardiac vascular ECs (protein) was markedly deceased, which validate the clinical relevance of our findings that endothelial PHD2 deficiency induces LV hypertrophy and cardiac fibrosis. Thus, targeting PHD2/HIF-2α signaling can be a therapeutic approach for pathological cardiac hypertrophy leading to cardiomyopathy and heart failure.

### Methods

### Human Samples and Data

The patient heart samples were collected from six cardiomyopathy patients including four hypertrophic cardiomyopathy, one dilated cardiomyopathy, and one hypertensive cardiomyopathy. Control samples were collected from similar age range individuals (five males, one female) without heart disease. The cause of death for controls was mechanical injuries. Failing heart samples were obtained from the left ventricular anterior wall during heart transplantation or implantation of LVAD (LV assistant device)^{21,22}. The non-failing heart samples were obtained from the LV free wall and procured from National Disease Research Interchange and University of Pennsylvania. Non-failing heart donors showed no laboratory signs of cardiac disease.

### Animals

Egln1^{Tie2Cre} mice, Egln1/Hif1a^{Tie2Cre} and Egln1/Hif2a^{Tie2Cre} double knockout mice were generated as described previously¹⁶. Egln1^{EndoCreERT2} mice were generated by crossing Egln1 floxed mice with EndoSCL-CreERT2 transgenic mice expressing the tamoxifen-inducible Cre recombinase under the control of the 5′ endothelial enhancer of the stem cell leukemia locus²³. At 8 weeks of age, Egln1^{EndoCreERT2} and Egln1^{f/f} mice were treated with 2 mg tamoxifen intraperitoneally for 5 days to induced Egln1 deletion only in ECs. Mice were scarified at the age of ˜9 months. Both male and female mice were used in these studies. The use of animals was in compliance with the guidelines of the Animal Care and Use Committee of the Northwestern University and of the University of Arizona.

### Echocardiography

Echocardiography was performed on a Fujifilm VisualSonics Vevo 2100 using an MS550D (40 MHz) transducer as described previously. Briefly, mice were anesthetized in an induction chamber filled with 1% isoflurane. The left ventricle anterior/posterior wall thickness during diastole (LVAW and LVPW), the LV internal dimension during diastole (LVID), the LV fractional shortening (LVFS) and the cardiac output (CO) were obtained from the parasternal short axis view using M-mode. Results were calculated using VisualSonics Vevo 2100 analysis software (v. 1.6) with a cardiac measurements package and were based on the average of at least three cardiac cycles.

### Reanalysis of Public Single-Cell RNA Sequencing Datasets

We used the publicly available metadata from two human fetal hearts (GSM4008686 and GSM4008687)²⁴. The metadata was processed in R (version 4.0.2) via Seurat package V3.2.3²⁵. Briefly, cells that expressed fewer than 100 genes, and cells that expressed over 4,000 genes, and cells with unique molecular identifiers (UMIs) more than 10% from the mitochondrial genome were filtered out. The data were normalized and integrated in Seurat, followed by Scaled and summarized by principal component analysis (PCA), and then visualized using UMAP plot. FindClusters function (resolution=0.5) in Seurat was used to cluster cells based on the gene expression profile. Cardiomyocyte (NPPA, TNNT2), endothelial cells (CDH5), fibroblasts (FN1, VIM), smooth muscle cells (ACTA2), and macrophages (CD68, CD14) clusters were annotated based on the expression of known markers.

### Irradiation and Bone Marrow Transplantation

WT or Egln1^{Tie2Cre} female mice at the age of 3 weeks were delivered at a dose of 750 cGy/mouse. At 3 hours following irradiation, mice were transplanted with 1×10⁷ bone marrow cells (in 150 μl of HBSS) isolated from male Egln1^{Tie2Cre} or WT mice through tail vein injection. Mice were used for heart dissection at the age of 3.5 months as described previously¹⁶

### Immunofluorescent, Immunohistochemical and Histological Staining

Following PBS perfusion, heart tissue was embedded in OCT for cryosectioning for immunofluorescent staining. Heart sections (5 μm) were fixed with 4% paraformaldehyde, followed by blocking with 0.1% Triton X-100 and 5% normal goat serum at room temperature for 1 h. After 3 washes, they were incubated with anti-Ki67 (1:25, Abcam, Cat #ab 1667), anti-CD31 antibody (1:25, BD Biosciences, Cat #550274) at 4° C. overnight and were then incubated with either Alexa 647-conjugated anti-rabbit IgG (Life Technology), or Alexa 594-conjugated anti-rabbit IgG or Alexa 488 conjugated anti-rat IgG at room temperature for 1 h after 3 washes. Nuclei were counterstained with DAPI contained in Prolong Gold mounting media (Life Technology).

For Wheat Germ Agglutinin (WGA) staining, WGA conjugated with FITC or WGA conjugated with Alexa 647 were stained with cryosectioned slides at room temple for 10 mins.

For immunohistochemistry staining on paraffin sections, the tissues were cut into 5 μm thick sections after paraffin processing. Heart sections were then dewaxed and dehydrated. Antigen retrieval was performed by boiling the slides in 10 mmol/L sodium citrate (pH 6.0) for 10 minutes. After blocking, slides were incubated with anti-PHD2 antibody (1:200, Cell Signaling Technology, Cat #4835), at 4° C. overnight. Slides were incubated with 6% H₂O₂ for 30 min after primary antibody incubation and were then biotinylated with a rabbit IgG and ABC kit (Vector Labs) for immunohistochemistry. The nucleus was co-stained with hematoxylin (Sigma-Aldrich).

For histological assessment, hearts were harvested and washed with PBS, followed by fixation in 4% formalin and dehydrated in 70% ethanol. After paraffin processing, the tissues were cut into semi-thin 5 m thick sections. Sections were stained with H & E staining or Masson's trichrome kit as a service of charge at Core Facility.

### QRT-PCR

Total RNA was isolated from frozen left ventricular tissues with Trizol reagents (Invitrogen) followed by purification with the RNeasy Mini kit including DNase I digestion (Qiagen). One microgram of RNA was transcribed into cDNA using the high-capacity cDNA reverse transcription kits (Applied Biosystems) according to the manufacturer's protocol. Quantitative RT-PCR analysis was performed on an ABI ViiA 7 Real-time PCR system (Applied Biosystems) with the FastStart SYBR Green Master kit (Roche Applied Science). Target mRNA was determined using the comparative cycle threshold method of relative quantitation. Cyclophilin was used as an internal control for analysis of expression of mouse genes. The primer sequences are provided in Table 1.

TABLE 1
QRT-PCR primers

| | | SEQ | | SEQ |
|---|---|---|---|---|
| Gene | Forward | ID | Reverse | ID |
| name | primer | NO: | primer | NO: |
| hEGLNI | AAAGACTGG | 1 | CTCGTGCTC | 2 |
| | GATGCCAA | | TCTCATCT | |
| | GGT | | GCA | |
| hGAPDH | GTCTCCTCTG | 3 | ACCACCCTG | 4 |
| | ACTTCAAC | | TTGCTGTA | |
| | AGCG | | GCCAA | |
| mAnp | GATAGATGAA | 5 | AGGATTGGA | 6 |
| | GGCAGGA | | GCCCAGAGT | |
| | AGCCGC | | GGACTAGG | |
| mBnp | TGTTTCTGCT | 7 | CTCCGACTT | 8 |
| | TTTCCTTTA | | TTCTCTTAT | |
| | TCTGTC | | CAGCTC | |
| mMyh7 | TGCAAAGGCT | 9 | GCCAACACC | 10 |
| | CCAGGTC | | AACCTGTC | |
| | TGAGGGC | | CAAGTTC | |
| mColal | TCACCAAACT | 11 | GACCAGGAG | 12 |
| | CAGAAGAT | | GACCAGG | |
| | GTAGGA | | AAG | |

### RNA-Sequencing

Total RNA was isolated from left ventricular tissues with Trizol reagents (Invitrogen) followed by purification with the RNeasy Mini kit including DNase I digestion (Qiagen). RNA sequencing was carried out by Novogene corporation on Illumina Hiseq platform. The original sequencing data were trimmed using FASTX and aligned to the reference genome using TopHat2. The differential expression analysis was performed using Cuffdiff software²⁶.

### Statistics

Statistical analysis of data was done with Prism 7 (GraphPad Software, Inc.). Statistical significance for multiple-group comparisons was determined by one-way ANOVA with Tukey post hoc analysis that calculates corrected P values. Two-group comparisons were analyzed by the unpaired two-tailed Student's t test. As the covariates may affect the response, two-group comparisons of data from samples of cardiopathy patients and controls were examined by linear regression models where the covariates including age were included/adjusted. All bar graphs represent means±SD.

### Results

Decreased PHD2 Expression in Patients with Cardiomyopathy

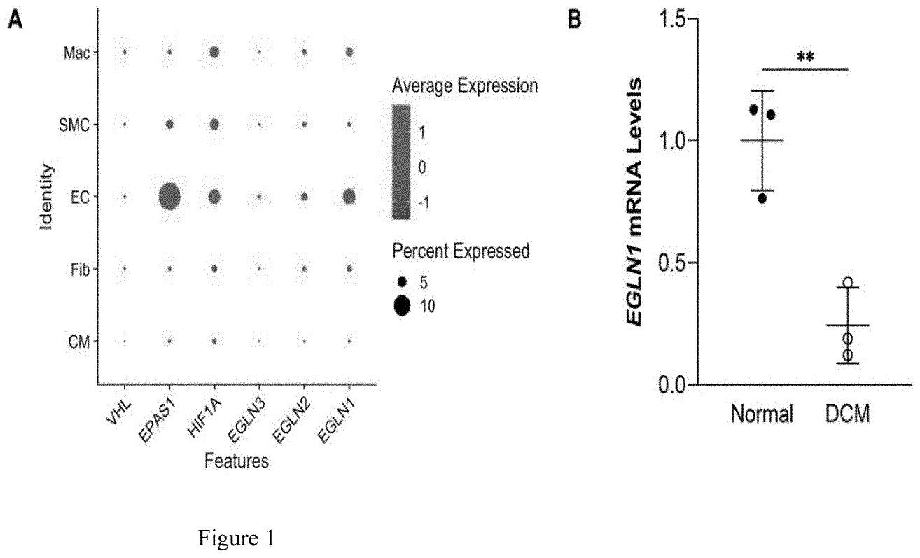

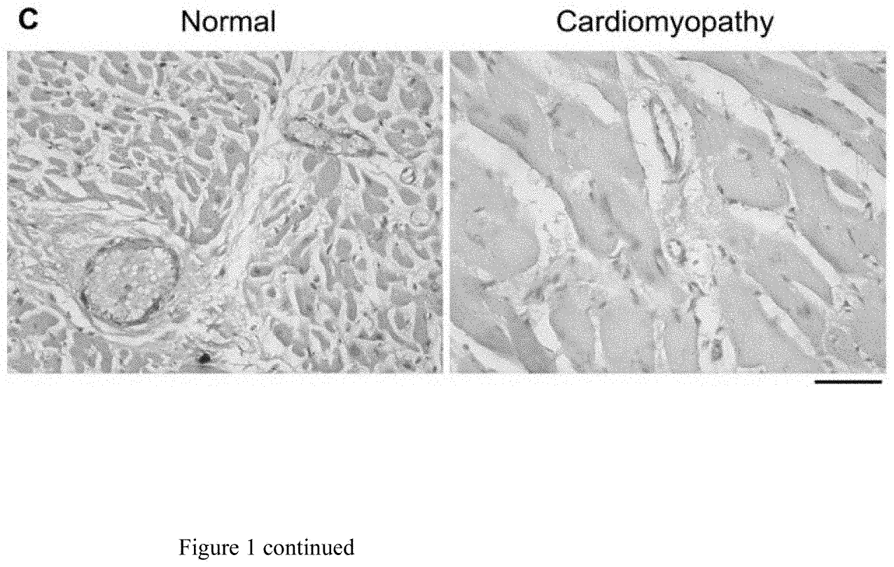

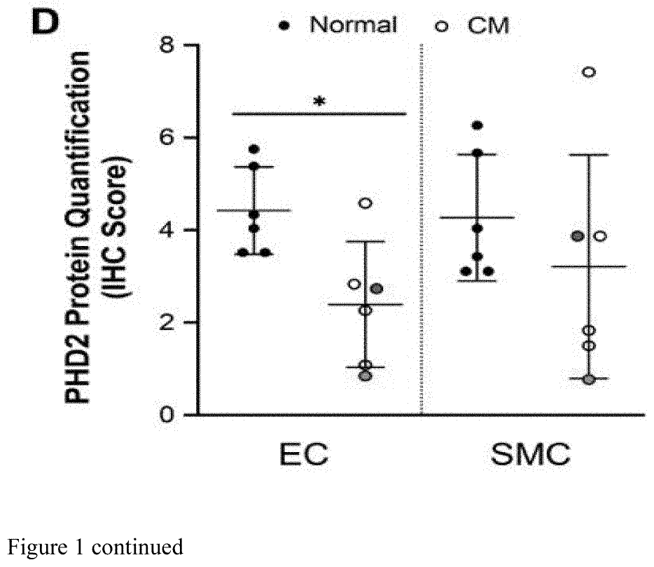

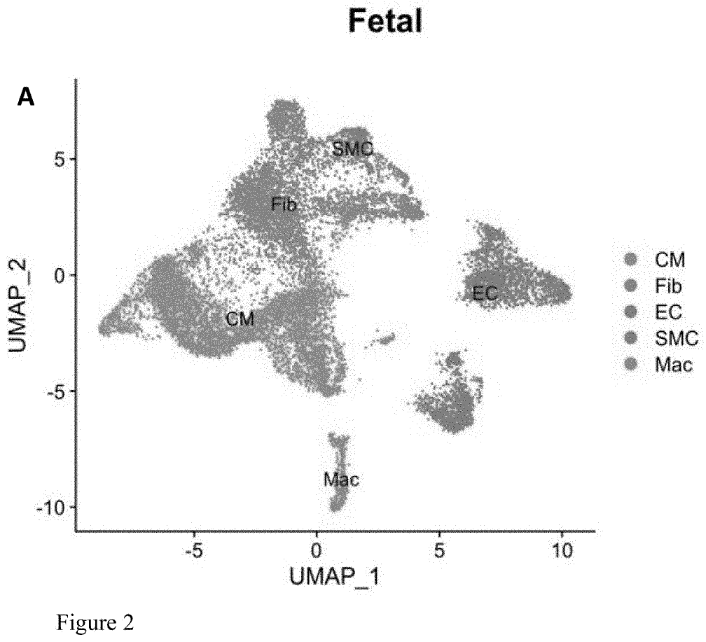

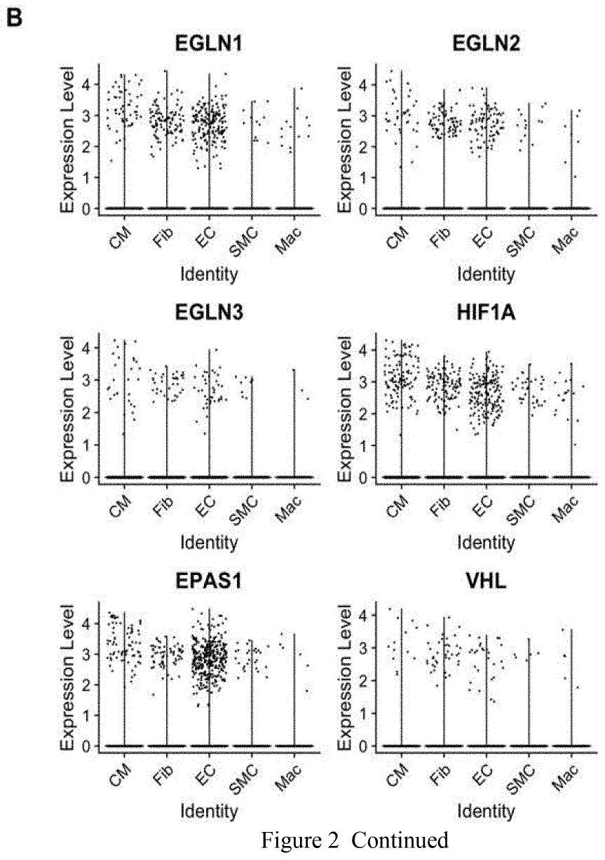

PHDs/VHL/HIF signaling have been implicated in many physiological and pathological conditions of heart development and heart diseases. Leveraging the public single-cell RNA-sequencing dataset, we first analyzed the mRNA expression of the key molecules of PHDs/VHL/HIF signaling from fetal and adult hearts. Our data demonstrated that EGLN1 and EPAS1 (encoding HIF-2α) are highly expressed in cardiac ECs in both the fetal and adult hearts (FIG. 1A and FIG. 2). We then examined the expression levels of PHD2 in heart tissues of cardiomyopathy patients and normal donors by quantitative (Q)RT-PCR. EGLN1 mRNA levels were drastically decreased in the LV cardiac tissue from patients with cardiomyopathy compared to normal donors (FIG. 1B). To further determine the cell-specific loss of PHD2 expression in the heart, we performed immunohistochemistry on LV heart sections. PHD2 was highly expressed in ECs as well as smooth muscle cells but only mildly in cardiomyocytes in normal donor hearts. However, its levels were dramatically reduced in cardiovascular ECs but not in smooth muscle cells of cardiomyopathy patients (FIGS. 1C and 1D). These data demonstrate a marked loss of endothelial PHD2 expression in patients with cardiomyopathy, suggesting a crucial role of endothelial PHD2 in cardiac function.

Constitutive loss of endothelial PHD2 induces left ventricular hypertrophy and fibrosis.

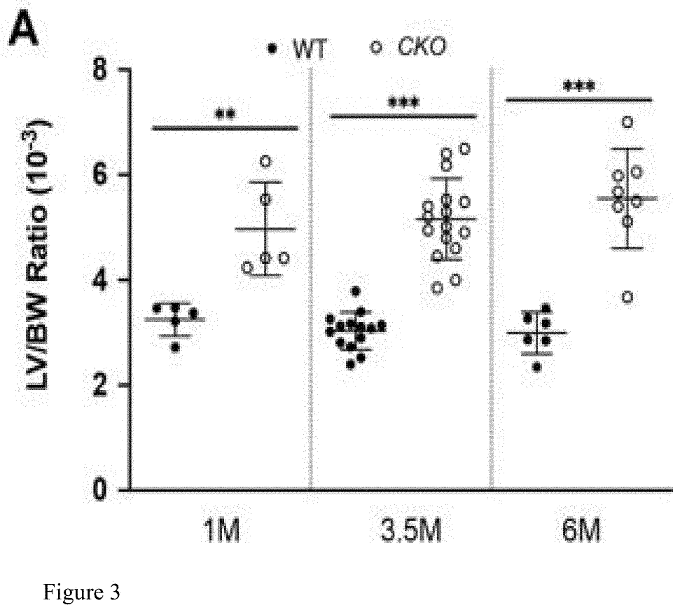

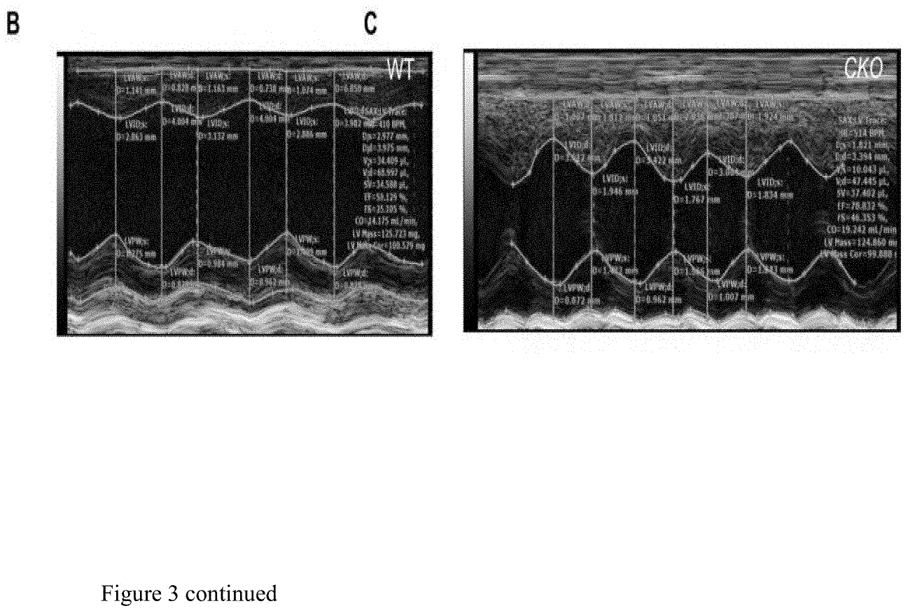

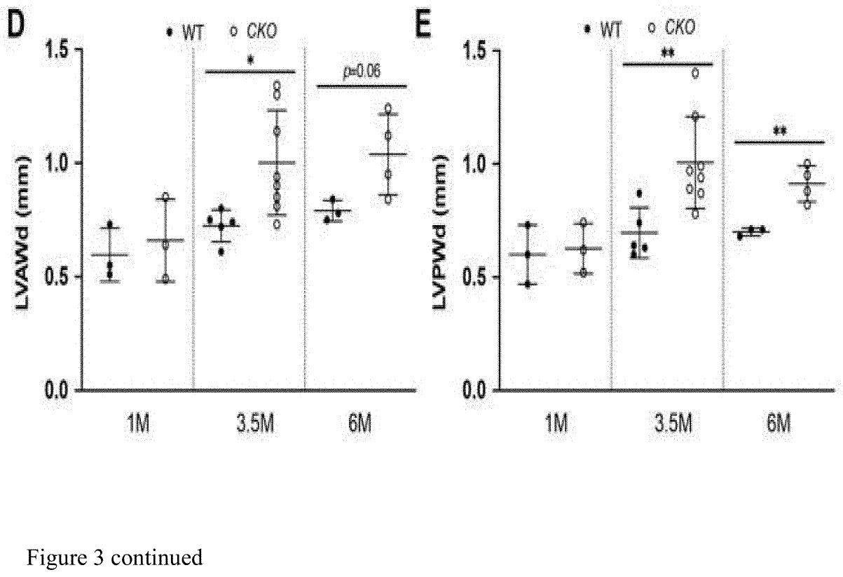

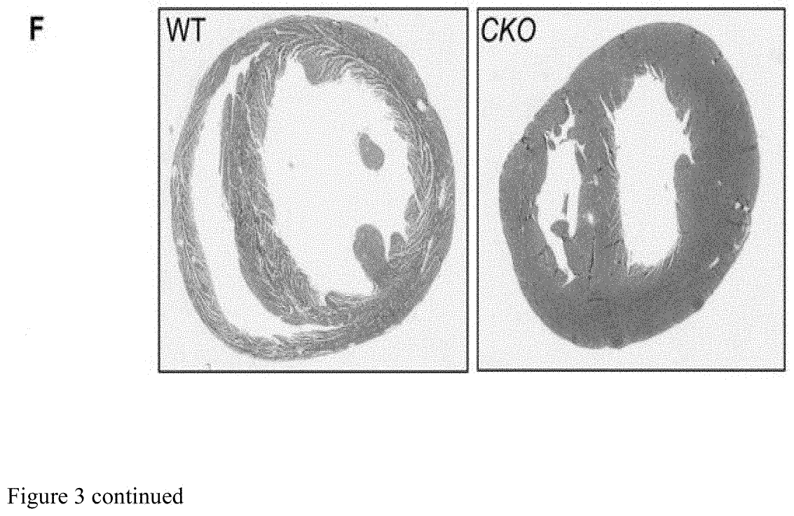

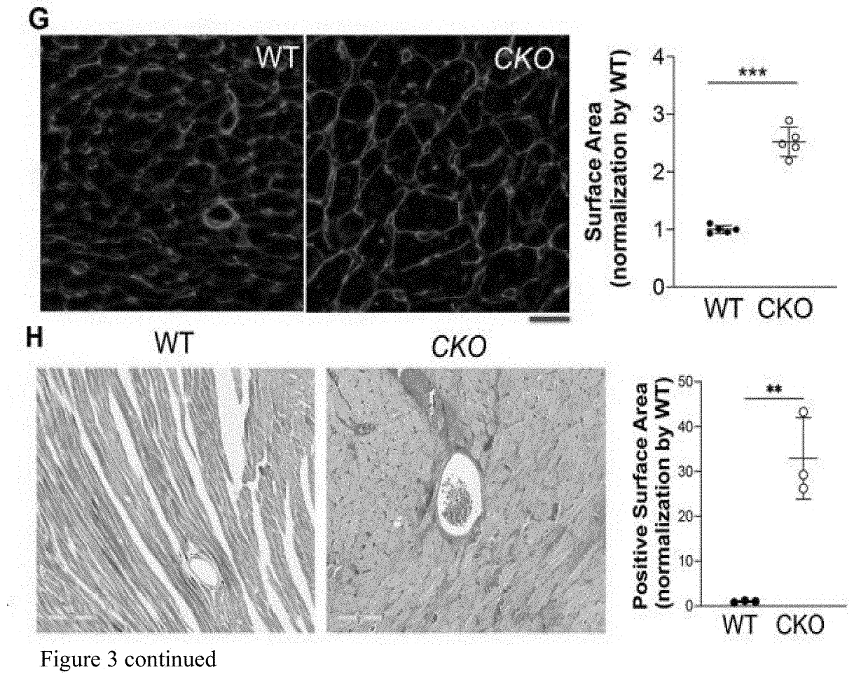

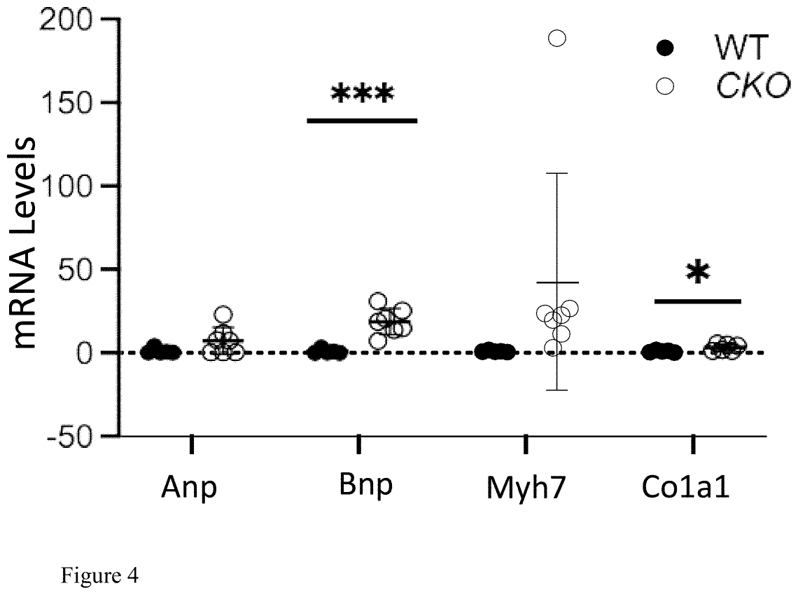

To investigate the role of endothelial PHD2 in the heart in vivo, we generated Egln1^{Tie2Cre} mice by breeding Egln1 floxed mice with Tie2Cre transgenic mice. Dissection of cardiac tissue showed marked increase of the ratio of LV vs body weight (LV/BW), indicative of LV hypertrophy (FIG. 3A). Echocardiography revealed marked increases of LV anterior and posterior wall thicknesses (FIG. 3B-3E). Histological examination demonstrated that Egln1^{Tie2Cre} mice exhibited marked increase of wall thickness of the LV as well as the RV and reduction of the chamber sizes, indicating cardiac hypertrophy (FIG. 3F). Wheat germ agglutinin (WGA) staining showed that LV cardiomyocytes from Egln1^{Tie2Cre} mice had marked increase of cellular surface, indicative of cardiomyocyte hypertrophy (FIG. 3G). QRT-PCR analysis revealed a marked increase of expression of Anp, Bnp and Myh7, further supporting cardiac hypertrophy (FIG. 4). We also observed significant perivascular and inter-cardiomyocyte fibrosis in the LV of Egln1^{Tie2Cre} mice by Trichrome staining (FIG. 31I). Consistently Colla expression was also markedly increased in the LV of Egln1^{Tie2Cre} mice (FIG. 4).

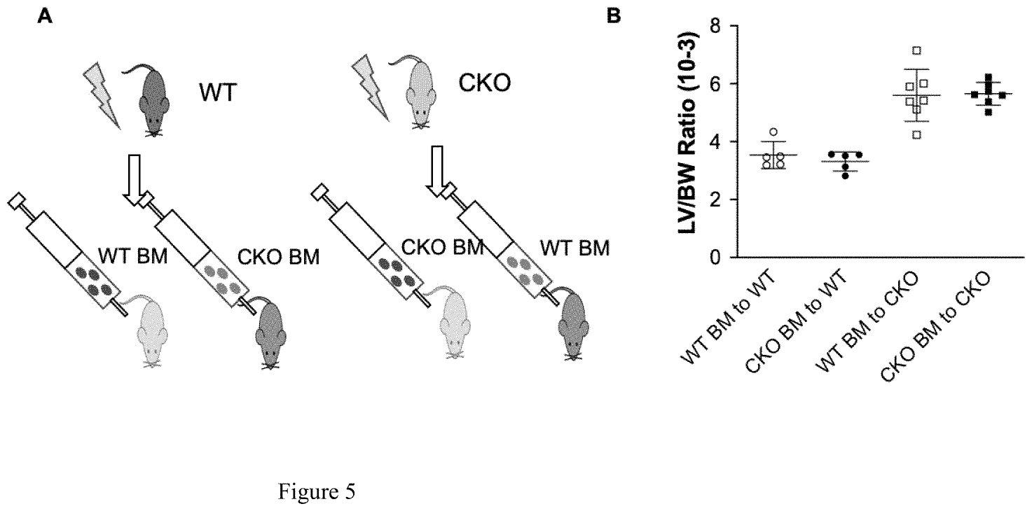

As Tie2Cre also expresses in hematopoietic cells in addition to ECs, we next determined whether hematopoietic cell-expressed PHD2 plays a role in inducing LV hypertrophy. We performed bone marrow transplantation via transplanting WT or Egln1^{Tie2Cre} bone marrow cells to lethally irradiated WT or Egln1^{Tie2Cre} mice. Three months post-transplantation, we did not observe any change of LV/BW ratio in WT mice transplanted with Egln1^{Tie2Cre} bone marrow cells compared to WT mice with WT bone marrow cells, indicating loss of PHD2 in bone marrow cells per se didn't induce LV hypertrophy. Similarly, WT bone marrow cell transplantation to Egln1^{Tie2Cre} mice didn't affect LV hypertrophy seen in Egln1^{Tie2Cre} mice transplanted with Egln1^{Tie2Cre} bone marrow cells (FIGS. 5A and 5B). These data suggest that loss of hematopoietic cell PHD2 is not involved in the development of LV hypertrophy.

### Inducible Deletion of Endothelial Egln1 in Adult Mice Leads to Development of LV Hypertrophy and Fibrosis

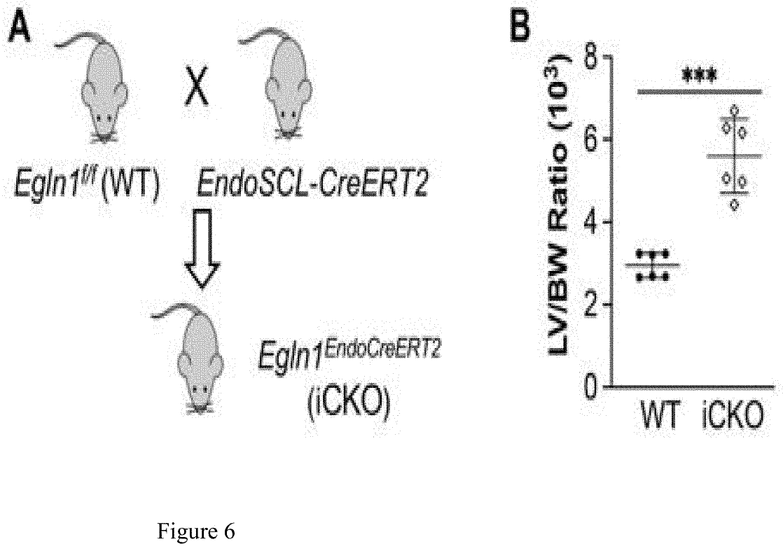

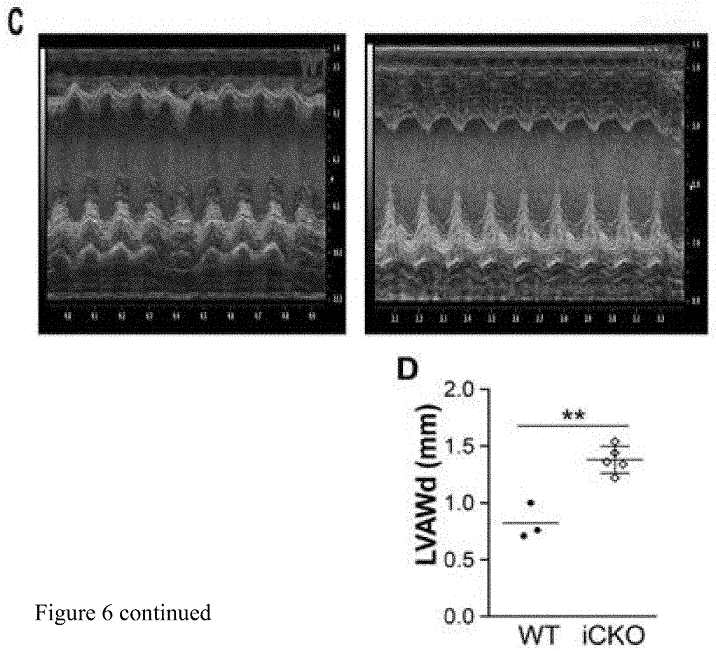

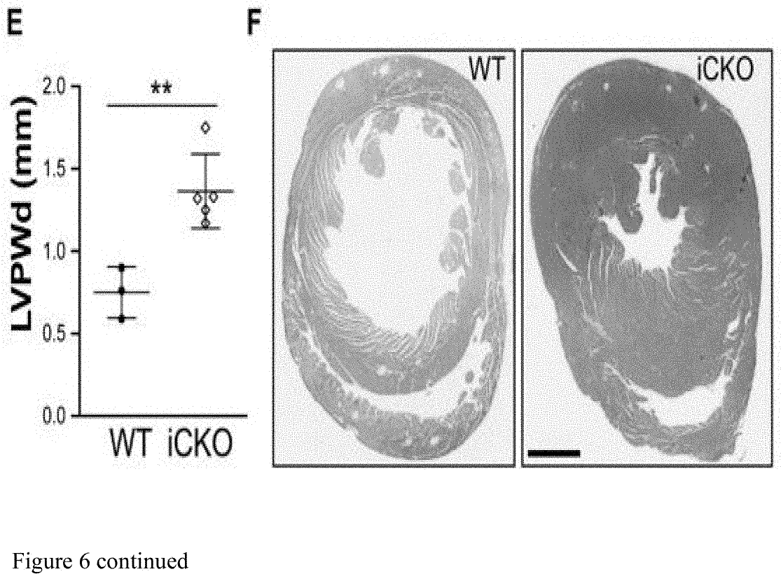

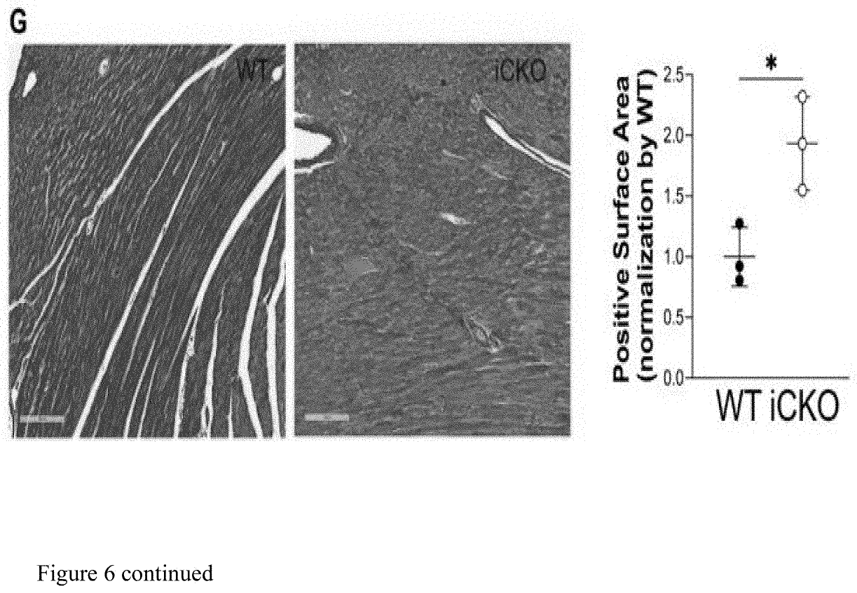

To determine if the cardiac hypertrophy in the Egln1^{Tie2Cre} mice is ascribed to potential developmental defects in mice with Tie2Cre-mediated deletion of Egln1, we generated mice with tamoxifen-inducible endothelial Egln1 deletion via breeding Egln1 floxed mice with EndoSCL-CreERT2 mice²⁷ (FIG. 6A), which induces EC-restricted gene disruption^{23,28-30}. Tamoxifen was administrated to Egln1^{EndoCreERT2} mice at age of 8 weeks. Seven months post-tamoxifen treatment, echocardiography, cardiac dissection, and histological analysis were employed to evaluate the cardiac phenotype. Egln1^{EndoCreERT2} mice exhibited a marked increase of LV/BW ratio (FIG. 6B), LV wall thicknesses and LV mass (FIG. 6C-6E). Histologic analysis also demonstrated marked increase of LV wall thickness, reduction of LV chamber size, and perivascular fibrosis by Trichrome staining (FIG. 6F-6G). However, the RV wall thickness and chamber size were not affected. Different from the Egln1^{Tie2Cre} mice by Tie2Cre-mediated deletion which also exhibit severe pulmonary hypertension^{16,31,32}, the Egln1^{EndoCreERT2} mice had only a mild increase of right ventricular systolic pressure (data not shown) indicative mild pulmonary hypertension consistent with minimal changes in RV wall thickness and chamber size. These data support the idea that loss of endothelial PHD2 in adult mice selectively induces LV hypertrophy.

### Increased Endothelial Proliferation and Angiogenesis in the LV of Endothelial PHD2-Deficient Mice

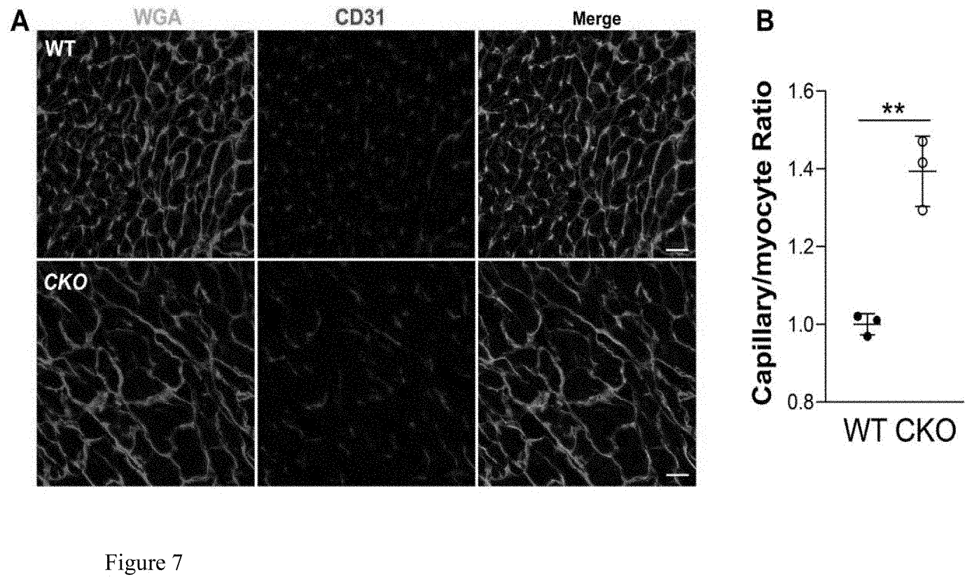

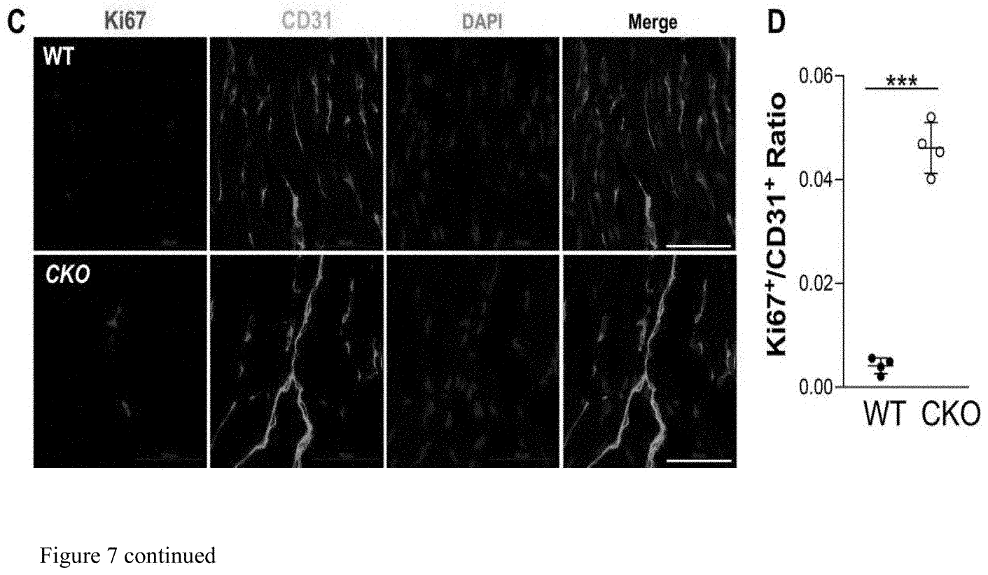

Previous studies demonstrated that stimulation of angiogenesis in the absence of other insults can drive myocardial hypertrophy in mice via overexpression of PR39 or VEGF-B⁵. PHD2 deficiency has been shown to induce EC proliferation and angiogenesis in vitro and in vivo^{33,34} To determine if vascular mass is increased in the LV of the Egln1^{Tie2Cre} mice, we performed immunostaining with endothelial marker CD31 and WGA. The capillary/myocyte ratio was drastically increased in the LV of Egln1^{Tie2Cre} mice compared to WT mice (FIGS. 7A and 7B). Anti-Ki67 immunostaining demonstrated a marked increase of Ki67+/CD31+ cells in the LV of Egln1^{Tie2Cre} mice (FIGS. 7C and 7D), suggesting that PHD2 deficiency induces EC proliferation in the LV, which explains the marked increase of capillary/myocyteratio.

### Distinct Role of Endothelial HIF-1α Versus HIF-2α in LV Hypertrophy

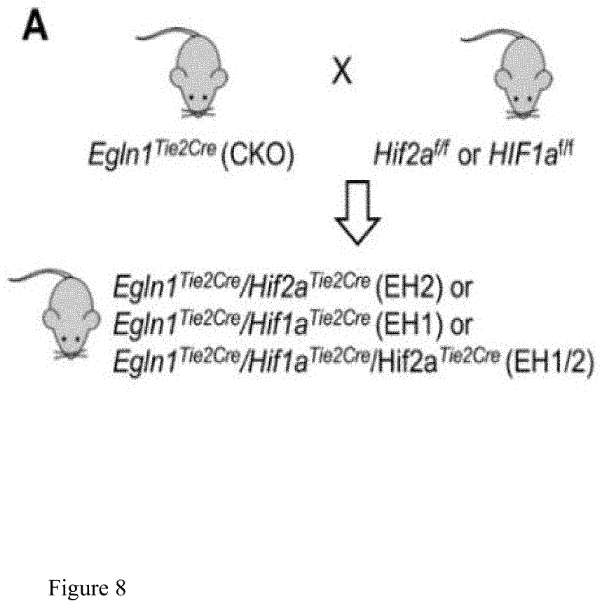

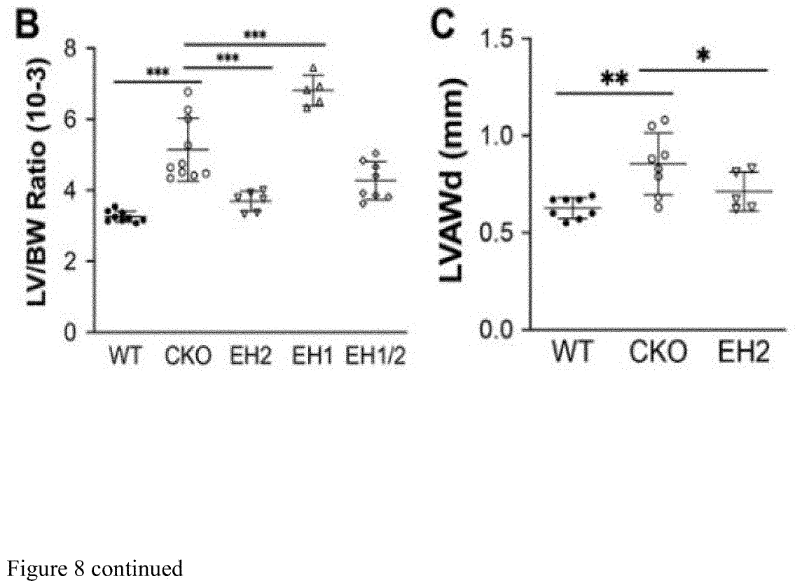

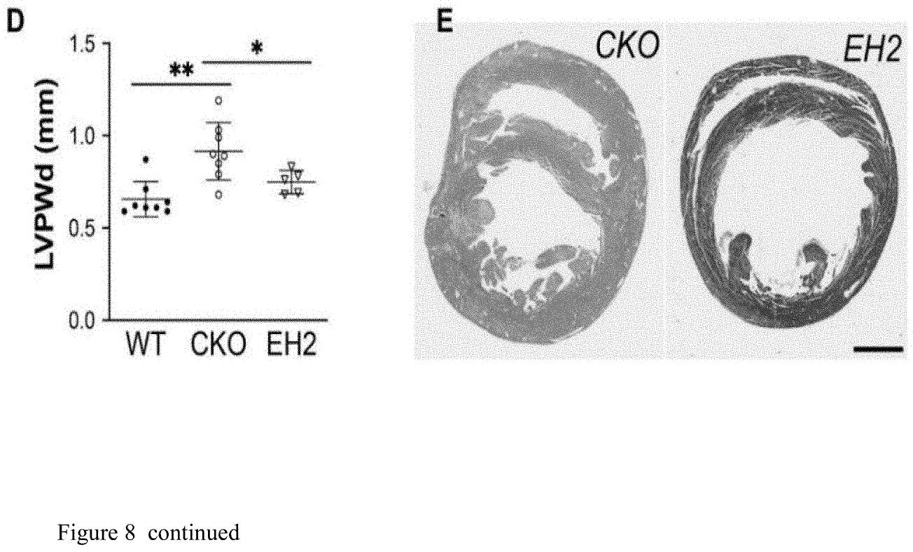

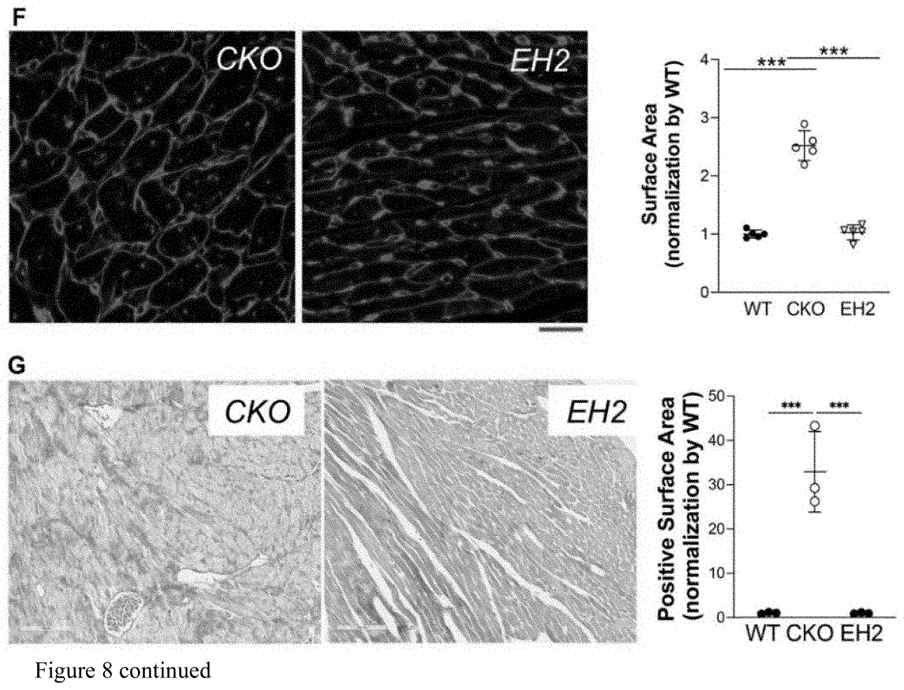

As Egln1 deletion stabilizes both HIF-1α and HIF-2α, we generated Egln1/Hif1a^{Tie2Cre} Egln1/Hif2a^{Tie2Cre} double knockout and Egln1/Hif1a/Hif2a^{Tie2Cre} triple knockout mice (FIG. 8A) to determine the HIF-α isoform(s) mediating LV hypertrophy. Heart dissection showed that Egln1/Hif2a^{Tie2Cre} and Egln1/Hif1a/Hif2a^{Tie2Cre} mice exhibited normal LV/BW ratio seen in WT mice, whereas Egln1/Hifla^{Tie2Cre} mice had increased LV/BW ratio compared to Egln1^{Tie2Cre} mice (FIG. 8B). These data provide in vivo evidence that endothelial HIF-2α activation secondary to loss of endothelial PHD2 is responsible for LV hypertrophy seen in Egln1^{Tie2Cre} mice whereas HIF-1α activation attenuates LV hypertrophy. Echocardiography confirmed that Egln1/Hif2a^{Tie2Cre} mice had normal LV wall thickness and LV mass in contrast to Egln1^{Tie2Cre} mice (FIGS. 8C and 8D). Histological assessment demonstrated that Egln1/Hif2a^{Tie2Cre} mice had no cardiac hypertrophy and fibrosis, as well as normal cardiomyocyte surface area (FIG. 8E-8G). These data demonstrate the causal role of endothelial HIF-2α activation in mediating LV hypertrophy and fibrosis seen in Egln1^{Tie2Cre} mice.

### RNA-Sequencing Analysis Identifies HIF-2α-Mediated Signaling Pathways Regulating Cardiac Hypertrophy and Fibrosis

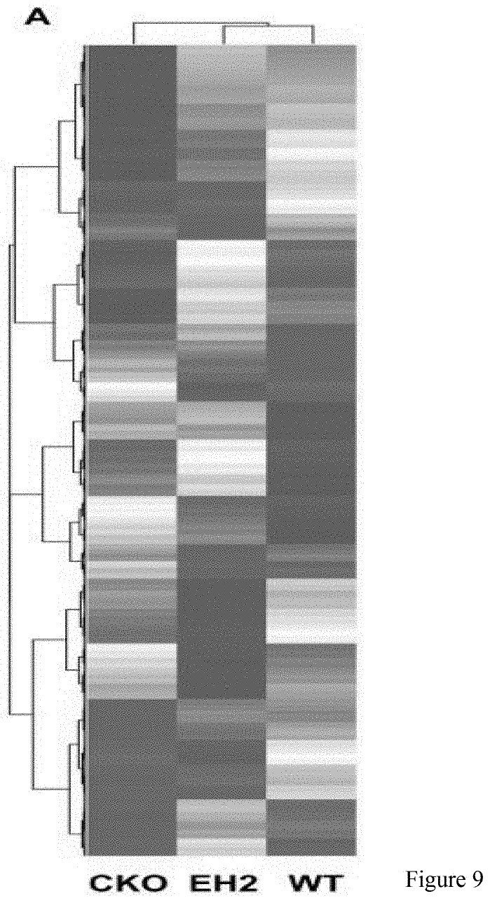

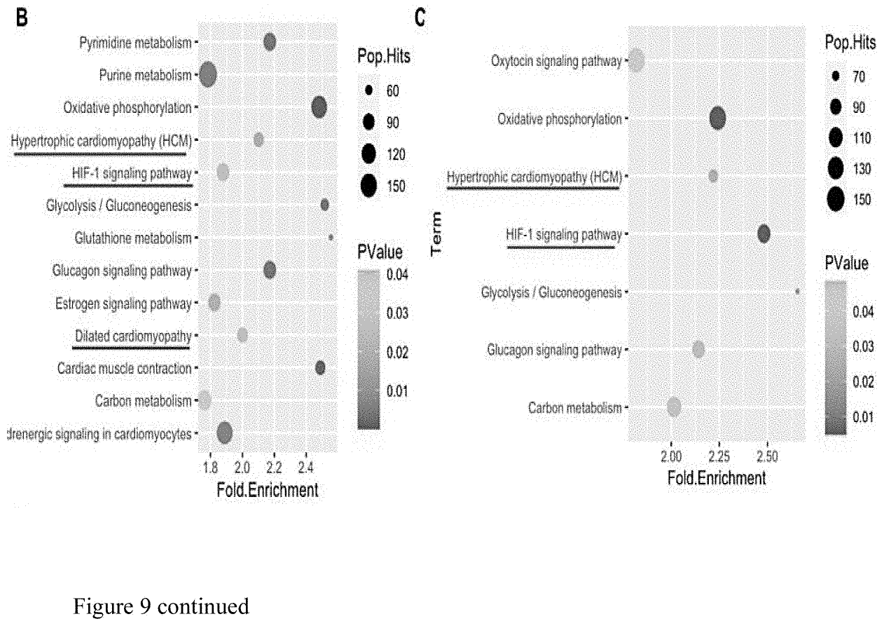

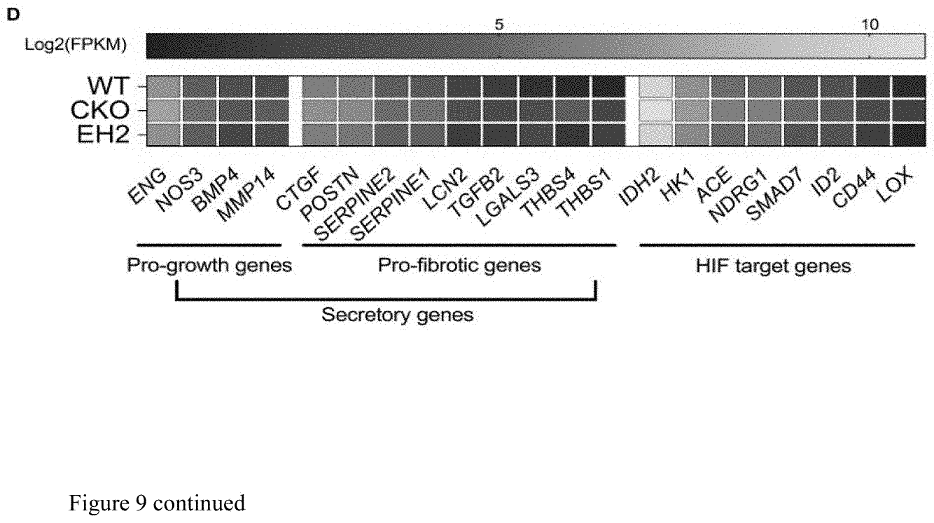

To understand the downstream mechanisms of endothelial HIF-2α in regulating cardiac function, we performed whole transcriptome RNA sequencing (RNA-seq) of LV tissue dissected from WT, Egln1^{Tie2Cre} and Egln1/Hif2a^{Tie2Cre} mice (FIG. 9A). Firstly, we found 1,454 genes (FPKM>5, fold change>1.5 or <0.66) were changed in Egln1^{Tie2Cre} LV compared to WT LV. Then, we performed the enriched Kyoto Encyclopedia of Genes and Genomes (KEGG) pathways analysis on the upregulated genes in Egln1^{Tie2Cre} vs WT mice. The analysis revealed that there were alterations of upregulated pathways related to adrenergic signaling in cardiomyocytes, hypertrophic cardiomyopathy, dilated cardiomyopathy, and cardiac muscle contraction, which are consistent with the hypertrophic phenotype we observed in Egln1^{Tie2Cre} mice (FIG. 9B). To determine the genes downstream of HIF-2α activation, we did the intersecting analysis of DEGs from WT_Egln1^{Tie2Cre} and Egln1^{Tie2Cre} Egln1/Hif2a^{Tie2Cre} There were 864 overlapping genes between WT_Egln1_{Tie2Cre} and Egln1^{Tie2Cre}_Egln1 Hif2a^{Tie2Cre}. KEGG pathway analysis of these genes upregulated in Egln1^{Tie2Cre} but normalized in Egln1/Hif2a^{Tie2Cre} hearts also showed enrichment of hypertrophic cardiomyopathy, and dilated cardiomyopathy (FIG. 9C), suggesting that these pathway abnormalities are downstream of HIF-2α activation. We also observed that many genes related to endothelium and EC-derived factors-mediated cell growth, and genes related to cardiac fibrosis were altered in Egln1^{Tie2Cre} mice whereas normalized in Egln1/Hif2a^{Tie2Cre} (FIG. 9D). These data provide mechanistic understanding of the distinct roles of endothelial HIF-1α and HIF-2α in regulating LV hypertrophy and fibrosis.

### Pharmacological Inhibition of HIF-2α Inhibits LV Hypertrophy

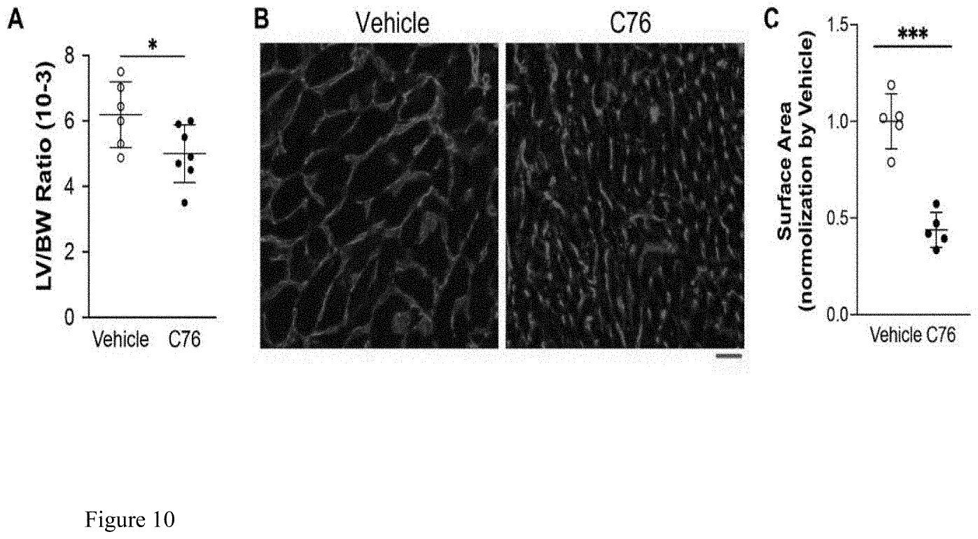

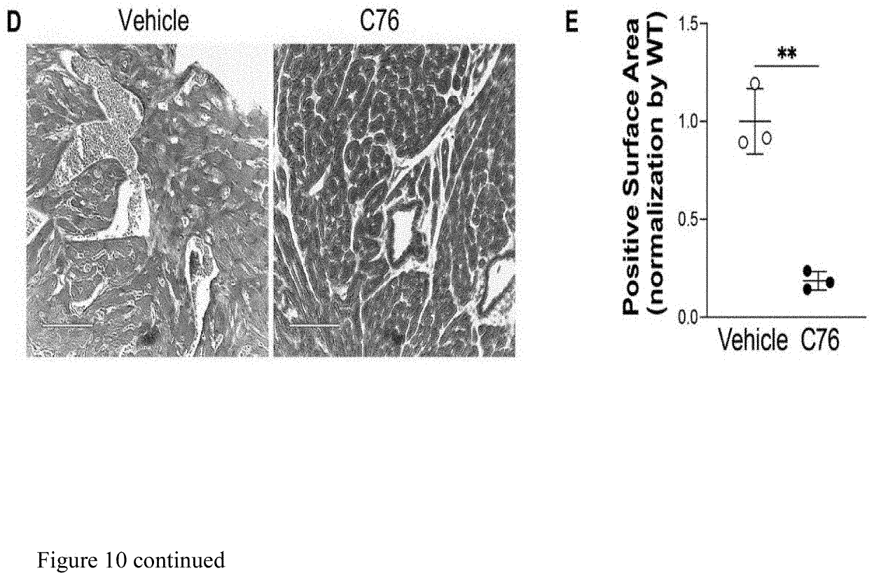

To assess the therapeutic potential of HIF-2α inhibition for cardiomyopathy, we treated 3 week-old Egln1^{Tie2Cre} mice with the HIF-2α translation inhibitor C76^{31,35} for 11 weeks. C76 treatment inhibited LV hypertrophy of Egln1^{Tie2Cre} mice evident by decreased LV/BW ratio compared to vehicle treatment (FIG. 10A). C76 treatment also significantly attenuated the cardiomyocyte surface area assessed by WGA staining (FIGS. 10B and 10C), and perivascular fibrosis by trichrome staining (FIGS. 10D and 10E). Taken together, these data demonstrated that inhibition of HIF-2α via a pharmacologic approach suppressed endothelial PHD2 deficiency-induced LV hypertrophy and cardiac fibrosis.

### Discussion

In this study we demonstrate that endothelial PHD2 is markedly decreased in the hearts of patients with cardiomyopathy. Genetic deletion of endothelial Egln1 in mice induces spontaneously severe cardiac hypertrophy and fibrosis. Through genetic deletion of Hif2a or Hif1a in Egln1^{Tie2Cre} mice, we also demonstrate that endothelial HIF-2α but not HIF-1α activation is responsible for PHD2 deficiency-induced LV hypertrophy and fibrosis. Moreover, pharmacological inhibition of HIF-2α reduces cardiac hypertrophy in Egln1^{Tie2Cre} mice. Thus, our data provide strong evidence that endothelial homeostasis is crucial to maintain normal cardiac function.

We demonstrate that PHD2 is mainly expressed in the vascular endothelium in human heart under normal condition, and its expression is markedly reduced in cardiac tissue and ECs of patients with cardiomyopathy.

Mice with Tie2Cre-mediated deletion of Egln1 develop LV hypertrophy and also RV hypertrophy associated with severe pulmonary hypertension^{16,31}. The RV hypertrophy is secondary to marked increase of pulmonary artery pressure. Furthermore, we employed the tamoxifen-inducible Egln1 knockout mice to determine the role of endothelial PHD2 deficiency in heart function. Seven months after tamoxifen treatment of 8 weeks old adult mice, we observed only LV hypertrophy in the mutant mice with quite normal RV size. These data provide clear evidence that endothelial PHD2 deficiency in adult mice selectively induces LV hypertrophy. Our study demonstrates that loss of endothelial PHD2 alone is sufficient to induce LV hypertrophy without marked changes in cardiomyocyte proliferation. We also observed marked LV cardiac fibrosis with predominantly in the peri-vascular regions and less in inter-cardiomyocytes area, which may lead to heart dysfunction. Cardiac hypertrophy is associated with fibrosis indicates maladaptive deleterious remodeling³⁶.

PHDs are oxygen sensors, which use molecular 02 as a substrate to hydroxylate proline residues of HIF-α. Deficiency of PHD2 results in stabilization and accumulation of HIF-α, and formation of HIF-α/HIF-β heterodimer, which consequently activates expression of a number of HIF target genes that regulate angiogenesis, inflammation and metabolism^{11,13,37} The cardiac remodeling phenotype in Egln1^{Tie2Cre} mice is ascribed to activation of HIF-2α but not HIF-1α, as Hif2a deletion in EC protects from Egln1-deficiency induced cardiac hypertrophy and fibrosis, whereas Hif1a deletion in Egln1^{Tie2Cre} mice does not show any protection but increases cardiac hypertrophy. Consistently, previous studies show that endothelial Hif1a deletion in TAC-challenged mice induce myocardial hypertrophy and fibrosis, and rapid decompensation³⁸. Although both HIF-1α and HIF-2α express in ECs and share similar DNA binding site, endothelial HIF-1α and HIF-2α plays quite distinct roles in cardiac homeostasis.

Increased vascular mass and EC proliferation are evident in the LV of Egln1^{Tie2Cre} mice demonstrating that PHD2 deficiency in cardiac vascular ECs induces EC proliferation and angiogenesis in mice. PHD2 deficiency in ECs induces angiogenesis which promotes cardiomyocyte hypertrophy leading to LV hypertrophy. Rarefaction of cardiac microvasculature is associated with pathological hypertrophy⁴⁰. We in fact observed a marked increase of capillary density in Egln1^{Tie2Cre} hearts, which may help to explain normal cardiac function including fractional shortening and ejection fraction in Egln1^{Tie2Cre} hearts

The heart is highly organized and consists of multiple cell types including cardiomyocytes, ECs and fibroblasts. Cell-cell communication in the heart is important for cardiac development and adaptation to stress such as pressure overload. It is well documented that soluble factors secreted by ECs maintain tissue homeostasis in different physiological and pathological microenvironment including cancer and bone marrow niche^{4,7,41}. To date, a growing number of cardio-active factors derived from ECs have been shown to regulate cardiac angiogenesis contributing to cardiac hypertrophy and/or dysfunction, including Nitric oxide (NO), Neuregulin-1, Basic fibroblast growth factor (FGF2), Platelet-derived growth factor (PDGF), Vascular endothelial growth factor (VEGF)^{8,41.} Other endothelium-derived factors such as Endothelin-1, NO, Neuregulin, Bmp4, Fgf23, Lgals3, Lcn2, Spp1, Tgfb1, can directly promote cardiac hypertrophy and fibrosis⁴²⁻⁴⁶. Our data suggest that endothelial-derived factors mediate EC-myocyte crosstalk to induce cardiac hypertrophy and fibrosis through excessive angiogenesis and/or paracrine effects.

Pathological cardiac hypertrophy worsens clinical outcomes and progresses to heart failure and death. Modulation of abnormal cardiac growth is becoming a potential approach for preventing and treating heart failure in patients². Our study demonstrates that pharmacological inhibition of HIF-2α reduces cardiac hypertrophy in Egln1^{Tie2Cre} mice, which provides strong evidence that targeting PHD2/HIF-2α signaling is a promising strategy for patients with pathological cardiac hypertrophy. The HIF-2α translation inhibitor C76 used in this study selectively inhibits HIF-2α translation by enhancing the binding of iron-regulatory protein 1 to the 5′-untranslated region of HIF2A mRNA without affecting HIF-1α expression^{31,35}.

In conclusion, our findings demonstrate for the first time that endothelial PHD2 deficiency in mice induces spontaneous cardiac hypertrophy and fibrosis via HIF-2α activation but not HIF-1α. PHD2 expression was markedly decreased in cardiovascular ECs of patients with cardiomyopathy, validating the clinical relevance of our findings in mice. Thus, selective targeting the abnormality of PHD2/HIF-2α signaling is a potential therapeutic strategy to treat patients with pathological cardiac hypertrophy and fibrosis.

### REFERENCE

1. Nakamura M, Sadoshima J. Mechanisms of physiological and pathological cardiac hypertrophy. Nat Rev Cardiol. 2018; 15:387-407.
2. Frey N, Olson E N. Cardiac Hypertrophy: The Good, the Bad, and the Ugly. Annu Rev Physiol. 2003; 65:45-79.
3. Gogiraju R, Schroeter M R, Bochenek M L, Hubert A, Münzel T, Hasenfuss G, Schäfer K. Endothelial deletion of protein tyrosine phosphatase-1B protects against pressure overload-induced heart failure in mice. Cardiovasc Res. 2016; 111:204-216.
4. Oka T, Akazawa H, Naito A T, Komuro I. Angiogenesis and Cardiac Hypertrophy. Circ Res. 2014; 114:565-571.
5. Tirziu D, Chorianopoulos E, Moodie K L, Palac R T, Zhuang Z W, Tjwa M, Roncal C, Eriksson U, Fu Q, Elfenbein A, Hall A E, Carmeliet P, Moons L, Simons M. Myocardial hypertrophy in the absence of external stimuli is induced by angiogenesis in mice. J Clin Invest. 2007; 117:3188-97.
6. Jaba I M, Zhuang Z W, Li N, Jiang Y, Martin K A, Sinusas A J, Papademetris X, Simons M, Sessa W C, Young L H, Tirziu D. No triggers rgs4 degradation to coordinate angiogenesis and cardiomyocyte growth. J Clin Invest. 2013; 123:1718-1731.
7. Shiojima I, Sato K, Izumiya Y, Schiekofer S, Ito M, Liao R, Colucci W S, Walsh K. Disruption of coordinated cardiac hypertrophy and angiogenesis contributes to the transition to heart failure. J Clin Invest. 2005; 115:2108-18.
8. Talman V, Kivelä R. Cardiomyocyte-Endothelial Cell Interactions in Cardiac Remodeling and Regeneration. Front Cardiovasc Med. 2018; 5:1-8.
9. Bassat E, Mutlak Y E, Genzelinakh A, Shadrin I Y, Baruch Umansky K, Yifa O, Kain D, Rajchman D, Leach J, Riabov Bassat D, Udi Y, Sarig R, Sagi I, Martin J F, Bursac N, Cohen S, Tzahor E. The extracellular matrix protein agrin promotes heart regeneration in mice. Nature. 2017; 547:179-184.
10. Epstein A C R, Gleadle J M, McNeill L A, Hewitson K S, O'Rourke J, Mole D R, Mukherji M, Metzen E, Wilson M I, Dhanda A, Tian Y M, Masson N, Hamilton D L, Jaakkola P, Barstead R, Hodgkin J, Maxwell P H, Pugh C W, Schofield C J, Ratcliffe P J. C. elegans EGL-9 and mammalian homologs define a family of dioxygenases that regulate HIF by prolyl hydroxylation. Cell. 2001; 107:43-54.
11. Semenza G L. Hypoxia-inducible factors in physiology and medicine. Cell. 2012; 148:399-408.
12. Ivan M, Kondo K, Yang H, Kim W, Valiando J, Ohh M, Salic A, Asara J M, Lane W S, Kaelin W G. HIFalpha targeted for VHL-mediated destruction by proline hydroxylation: implications for O2 sensing. Science. 2001; 292:464-8.
13. Majmundar A J, Wong W J, Simon M C. Hypoxia-inducible factors and the response to hypoxic stress. Mol Cell. 2010; 40:294-309.
14. Lee K E, Simon M C. SnapShot: Hypoxia-Inducible Factors. Cell. 2015; 163:1288-1288.el.
15. Wiesener M S, Jürgensen J S, Rosenberger C, Scholze C, Hörstrup J H, Warnecke C, Mandriota S, Bechmann I, Frei U A, Pugh C W, Ratcliffe P J, Bachmann S, Maxwell P H, Eckardt K. Widespread, hypoxia-inducible expression of HIF-2α in distinct cell populations of different organs. FASEB J. 2003; 17:271-273.
16. Dai Z, Li M, Wharton J, Zhu M M, Zhao Y Y. Prolyl-4 Hydroxylase 2 (PHD2) deficiency in endothelial cells and hematopoietic cells induces obliterative vascular remodeling and severe pulmonary arterial hypertension in mice and humans through hypoxia-inducible factor-2α. Circulation. 2016; 133:2447-2458.
17. Huang X, Zhang X, Zhao D X, Yin J, Hu G, Evans C E, Zhao Y Y. Endothelial Hypoxia-Inducible Factor-1α Is Required for Vascular Repair and Resolution of Inflammatory Lung Injury through Forkhead Box Protein M1. Am J Pathol. 2019; 189:1664-1679.
18. Minamishima Y A, Moslehi J, Bardeesy N, Cullen D, Bronson R T, Kaelin W G. Somatic inactivation of the PHD2 prolyl hydroxylase causes polycythemia and congestive heart failure. Blood. 2008; 111:3236-3244.
19. Holscher M, Silter M, Krull S, Von Ahlen M, Hesse A, Schwartz P, Wielockx B, Breier G, Katschinski D M, Zieseniss A. Cardiomyocyte-specific prolyl-4-hydroxylase domain 2 knock out protects from acute myocardial ischemic injury. J Biol Chem. 2011; 286:11185-11194.
20. Fan Q, Mao H, Angelini A, Coarfa C, Robertson M J, Lagor W R, Wehrens X H T, Martin J F, Pi X, Xie L. Depletion of Endothelial Prolyl Hydroxylase Domain Protein 2 and 3 Promotes Cardiomyocyte Proliferation and Prevents Ventricular Failure Induced by Myocardial Infarction. Circulation. 2019; 140:440-442.
21. Gao C, Ren S V, Yu J, Baal U, Thai D, Lu J, Zeng C, Yan H, Wang Y. Glucagon Receptor Antagonism Ameliorates Progression of Heart Failure. JACC Basic to Transl Sci. 2019; 4:161-172.
22. Wang Z, Zhang X J, Ji Y X, Zhang P, Deng K Q, Gong J, Ren S, Wang X, Chen I, Wang H, Gao C, Yokota T, Ang Y S, Li S, Cass A, Vondriska T M, Li G, Deb A, Srivastava D, Yang H T, Xiao X, Li H, Wang Y. The long noncoding RNA Chaer defines an epigenetic checkpoint in cardiac hypertrophy. Nat Med. 2016; 22:1131-1139.
23. Tran K A, Zhang X, Predescu D, Huang X, Machado R F, Gothert J R, Malik A B, Valyi-Nagy T, Zhao Y-Y. Endothelial β-Catenin Signaling Is Required for Maintaining Adult Blood-Brain Barrier Integrity and Central Nervous System Homeostasis. Circulation. 2016; 133:177-186.
24. Han X, Zhou Z, Fei L, Sun H, Wang R, Chen Y, Chen H, Wang J, Tang H, Ge W, Zhou Y, Ye F, Jiang M, Wu J, Xiao Y, Jia X, Zhang T, Ma X, Zhang Q, Bai X, Lai S, Yu C, Zhu L, Lin R, Gao Y, Wang M, Wu Y, Zhang J, Zhan R, Zhu S, Hu H, Wang C, Chen M, Huang H, Liang T, Chen J, Wang W, Zhang D, Guo G. Construction of a human cell landscape at single-cell level. Nature. 2020; 581:303-309.
25. Stuart T, Butler A, Hoffman P, Hafemeister C, Papalexi E, Mauck W M, Hao Y, Stoeckius M, Smibert P, Satija R. Comprehensive Integration of Single-Cell Data. Cell. 2019; 177:1888-1902.e21.
26. Chen J, Sathiyamoorthy K, Zhang X, Schaller S, Perez White B E, Jardetzky T S, Longnecker R. Ephrin receptor A2 is a functional entry receptor for Epstein-Barr virus. Nat Microbiol. 2018; 3:172-180.
27. Gothert J R, Gustin S E, Van Eekelen J A M, Schmidt U, Hall M A, Jane S M, Green A R, Gottgens B, Izon D J, Begley C G. Genetically tagging endothelial cells in vivo: Bone marrow-derived cells do not contribute to tumor endothelium. Blood. 2004; 104:1769-1777.
28. Nussbaum C, Bannenberg S, Keul P, Graler M H, Gonsalves-De-Albuquerque C F, Korhonen H, Von Wnuck Lipinski K, Heusch G, De Castro Faria Neto H C, Rohwedder I, Gothert J R, Prasad V P, Haufe G, Lange-Sperandio B, Offermanns S, Sperandio M, Levkau B. Sphingosine-1-phosphate receptor 3 promotes leukocyte rolling by mobilizing endothelial P-selectin. Nat Commun. 2015; 6.
29. Weis S M, Lim S T, Lutu-Fuga K M, Barnes L A, Chen X L, Gothert J R, Shen T L, Guan J L, Schlaepfer D D, Cheresh D A. Compensatory role for Pyk2 during angiogenesis in adult mice lacking endothelial cell FAK. J Cell Biol. 2008; 181:43-50.
30. Cheng K T, Xiong S, Ye Z, Hong Z, Di A, Tsang K M, Gao X, An S, Mittal M, Vogel S M, Miao E A, Rehman J, Malik A B. Caspase-11-mediated endothelial pyroptosis underlies endotoxemia-induced lung injury. J Clin Invest. 2017; 127:4124-4135.
31. Dai Z, Zhu M M, Peng Y, Machireddy N, Evans C E, Machado R, Zhang X, Zhao Y Y. Therapeutic targeting of vascular remodeling and right heart failure in pulmonary arterial hypertension with a HIF-2α inhibitor. Am J Respir Crit Care Med. 2018; 198:1423-1434.
32. Dai Z, Zhu M M, Peng Y, Jin H, Machireddy N, Qian Z, Zhang X, Zhao Y Y. Endothelial and smooth muscle cell interaction via FoxMl signaling mediates vascular remodeling and pulmonary hypertension. Am J Respir Crit Care Med. 2018; 198:788-802.
33. Wu S, Nishiyama N, Kano M R, Morishita Y, Miyazono K, Itaka K, Chung U II, Kataoka K. Enhancement of angiogenesis through stabilization of hypoxia-inducible factor-1 by silencing prolyl hydroxylase domain-2 gene. Mol Ther. 2008; 16:1227-1234.
34. Chan D A, Kawahara T L A, Sutphin P D, Chang H Y, Chi J T, Giaccia A J. Tumor Vasculature Is Regulated by PHD2-Mediated Angiogenesis and Bone Marrow-Derived Cell Recruitment. Cancer Cell. 2009; 15:527-538.
35. Zimmer M, Ebert B L, Neil C, Brenner K, Papaioannou I, Melas A, Tolliday N, Lamb J, Pantopoulos K, Golub T, Iliopoulos O. Small-molecule inhibitors of HIF-2a translation link its 5′UTR iron-responsive element to oxygen sensing. Mol Cell. 2008; 32:838-48.
36. Frey N, Katus H A, Olson E N, Hill J A. Hypertrophy of the Heart: A New Therapeutic Target?Circulation. 2004; 109:1580-1589.
37. Bishop T, Ratcliffe P J. HIF Hydroxylase Pathways in Cardiovascular Physiology and Medicine. Circ Res. 2015; 117:65-79.
38. Wei H, Bedja D, Koitabashi N, Xing D, Chen J, Fox-Talbot K, Rouf R, Chen S, Steenbergen C, Harmon J W, Dietz H C, Gabrielson K L, Kass D A, Semenza G L. Endothelial expression of hypoxia-inducible factor 1 protects the murine heart and aorta from pressure overload by suppression of TGF-β signaling. Proc Natl Acad Sci USA. 2012; 109: E841-50.
39. Takeda K, Fong G H. Prolyl hydroxylase domain 2 protein suppresses hypoxia-induced endothelial cell proliferation. Hypertension. 2007; 49:178-184.
40. Mohammed S F, Hussain S, Mirzoyev S A, Edwards W D, Maleszewski J J, Redfield M M. Coronary microvascular rarefaction and myocardial fibrosis in heart failure with preserved ejection fraction. Circulation. 2015; 131:550-559.
41. Gogiraju R, Bochenek M L, Schafer K. Angiogenic Endothelial Cell Signaling in Cardiac Hypertrophy and Heart Failure. Front Cardiovasc Med. 2019; 6.42. Grabner A, Schramm K, Silswal N, Hendrix M, Yanucil C, Czaya B, Singh S, Wolf M, Hermann S, Stypmann J, Marco G S D I, Brand M, Wacker M J, Faul C. FGF23/FGFR4-mediated left ventricular hypertrophy is reversible. Sci Rep. 2017; 7:1-12.
43. Sun B, Huo R, Sheng Y, Li Y, Xie X, Chen C, Liu H Bin, Li N, Li C B, Guo W T, Zhu J X, Yang B F, Dong D L. Bone morphogenetic protein-4 mediates cardiac hypertrophy, apoptosis, and fibrosis in experimentally pathological cardiac hypertrophy. Hypertension. 2013; 61:352-360.
44. de Boer R A, Edelmann F, Cohen-Solal A, Mamas M A, Maisel A, Pieske B. Galectin-3 in heart failure with preserved ejection fraction. Eur J Heart Fail. 2013; 15:1095-1101.
45. Khalil H, Kanisicak O, Prasad V, Correll R N, Fu X, Schips T, Vagnozzi R J, Liu R, Huynh T, Lee S-J, Karch J, Molkentin J D. Fibroblast-specific TGF-β-Smad2/3 signaling underlies cardiac fibrosis. J Clin Invest. 2017; 127:3770-3783.
46. Lopez B, Gonzalez A, Lindner D, Westermann D, Ravassa S, Beaumont J, Gallego I, Zudaire A, Brugnolaro C, Querej eta R, Larman M, Tschöpe C, Diez J. Osteopontin-mediated myocardial fibrosis in heart failure: A role for lysyl oxidase? Cardiovasc Res. 2013; 99:111-120.
47. Cho H, Du X, Rizzi J P, Liberzon E, Chakraborty A A, Gao W, Carvo I, Signoretti S, Bruick R K, Josey J A, Wallace E M, Kaelin W G. On-target efficacy of a HIF-2α antagonist in preclinical kidney cancer models. Nature. 2016; 539:107-111.
48. Chen W, Hill H, Christie A, Kim M S, Holloman E, Pavia-Jimenez A, Homayoun F, Ma Y, Patel N, Yell P, Hao G, Yousuf Q, Joyce A, Pedrosa I, Geiger H, Zhang H, Chang J, Gardner K H, Bruick R K, Reeves C, Hwang T H, Courtney K, Frenkel E, Sun X, Zojwalla N, Wong T, Rizzi J P, Wallace E M, Josey J A, Xie Y, Xie X J, Kapur P, McKay R M, Brugarolas J. Targeting renal cell carcinoma with a HIF-2 antagonist. Nature. 2016; 539:112-117.
49. Courtney K D, Infante J R, Lam E T, Figlin R A, Rini B I, Brugarolas J, Zojwalla N J, Lowe A M, Wang K, Wallace E M, Josey J A, Choueiri T K. Phase I Dose-Escalation Trial of PT2385, a First-in-Class Hypoxia-Inducible Factor-2α Antagonist in Patients With Previously Treated Advanced Clear Cell Renal Cell Carcinoma. J Clin Oncol. 2018; 36:867-874.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating cardiovascular disease, the method comprising administering to the subject in need an effective amount of a HIF-2α inhibitor.

2. The method of claim 1, wherein the cardiovascular disease is cardiomyopathy, left ventricle (LV) hypertrophy or heart failure.

3. The method of claim 1, wherein the cardiovascular disease is cardiomyopathy and heart failure.

4. The method of claim 1, wherein the cardiovascular disease is characterized by a decreased expression of PHD2 in endothelial cells.

5. The method of claim 1, wherein the HIF-2α inhibitor is selected ethacridine, quinidine, hydroxyethylapoquinine, hydroxyethylquinine; hydroxyethylquinine, methantheline, propantheline, an analog thereof, or any combination thereof.

6. The method of claim 5, wherein the cardiovascular disease is cardiomyopathy and heart failure.

7. The method of claim 1, wherein the HIF-2α inhibitor is selected from of PT2977, PT2385, PT2399, NKT2152, C76, an analog thereof, or any combination thereof.

8. The method of claim 7, wherein the cardiovascular disease is cardiomyopathy and heart failure.

9. The method of claim 1, wherein the HIF-2α inhibitor is PHD2.

10. The method of claim 1, wherein the method further comprises administering an agent to activate PHD2 or induce PHD2 expression.

11. A method of reducing left ventricular hypertrophy, wherein the method comprises administering an effective amount of a HIF-2α inhibitor.

12. The method of claim 11, wherein the HIF-2α inhibitor is selected ethacridine, quinidine, hydroxyethylapoquinine, hydroxyethylquinine; hydroxyethylquinine, methantheline, propantheline, an analog thereof, or any combination thereof.

13. The method of claim 11, wherein the HIF-2α inhibitor is selected from of PT2977, PT2385, PT2399, NKT2152, C76, an analog thereof, or any combination thereof.

14. The method of claim 11, wherein the method further comprises administering an agent to activate PHD2 or induce PHD2 expression.

15. A method for treating cardiovascular disease, wherein the method comprises administering an effective amount of an agent to activate PHD2 or to induce PHD2 expression.

16. The method of claim 15, wherein the cardiovascular disease is cardiomyopathy, LV hypertrophy or heart failure.

17. The method of claim 15, wherein the cardiovascular disease is cardiomyopathy and heart failure.

18. The method of claim 15, wherein the cardiovascular disease is LV hypertrophy.

19. The method of claim 15, wherein the cardiovascular disease is characterized by a decreased expression of PHD2 in endothelial cells.

20. The method of claim 15, wherein the agent is selected from KRH102053 or analogs thereof or KRH102140 or analogs thereof.


SEQUENCE LISTING (12 sequences)

SEQ ID NO: 1 - DNA, 20 bp, synthetic construct
  source 1..20: mol_type = other DNA
aaagactgggatgccaaggt

SEQ ID NO: 2 - DNA, 20 bp, synthetic construct
  source 1..20: mol_type = other DNA
ctcgtgctctctcatctgca

SEQ ID NO: 3 - DNA, 22 bp, synthetic construct
  source 1..22: mol_type = other DNA
gtctcctctgacttcaacagcg

SEQ ID NO: 4 - DNA, 22 bp, synthetic construct
  source 1..22: mol_type = other DNA
accaccctgttgctgtagccaa

SEQ ID NO: 5 - DNA, 23 bp, synthetic construct
  source 1..23: mol_type = other DNA
gatagatgaaggcaggaagccgc

SEQ ID NO: 6 - DNA, 26 bp, synthetic construct
  source 1..26: mol_type = other DNA
aggattggagcccagagtggactagg

SEQ ID NO: 7 - DNA, 25 bp, synthetic construct
  source 1..25: mol_type = other DNA
tgtttctgcttttcctttatctgtc

SEQ ID NO: 8 - DNA, 24 bp, synthetic construct
  source 1..24: mol_type = other DNA
ctccgacttttctcttatcagctc

SEQ ID NO: 9 - DNA, 24 bp, synthetic construct
  source 1..24: mol_type = other DNA
tgcaaaggctccaggtctgagggc

SEQ ID NO: 10 - DNA, 24 bp, synthetic construct
  source 1..24: mol_type = other DNA
gccaacaccaacctgtccaagttc

SEQ ID NO: 11 - DNA, 24 bp, synthetic construct
  source 1..24: mol_type = other DNA
tcaccaaactcagaagatgtagga

SEQ ID NO: 12 - DNA, 19 bp, synthetic construct
  source 1..19: mol_type = other DNA
gaccaggaggaccaggaag